(19) United States
(12) Patent Application Publication
(10) Pub. No.: US 2009/0081183 A1
(43) Pub. Date: Mar. 26, 2009

(54) HIV Transcription Repressor Complex and Compositions and Methods Based Thereon

(75) Inventors: David M. Margolis, Dallas, TX (US); Fabio Romerio, Baltimore, MD (US); Jason J. Coull, Dallas, TX (US)

Correspondence Address: MYERS BIGEL SIBLEY & SAJOVEC, PO BOX 37428, RALEIGH, NC, 27627, US

(73) Assignee: The University of North Carolina at Chapel Hill

(21) Appl. No.: 12/032,043

(22) Filed: Feb. 15, 2008

Related U.S. Application Data
(63) Continuation of application No. 09/611,949, filed on Jul. 6, 2000, now abandoned.
Application No. 09/611,949 is a continuation-in-part of application No. 09/355,010, filed on Dec. 8, 1999, now abandoned.
(60) Provisional application No. 60/036,242, filed on Jan. 23, 1997.

(51) Int. Cl. A61K 38/16; A61K 38/50; A61P 31/18

(52) U.S. Cl. 424/94.6; 514/12

(57) ABSTRACT

The molecular mechanism of YY1/LSF-associated repression of HIV-1 is described herein. More particularly, an HIV transcription repressor complex containing YY1, LSF and HDAC1 is described. The invention is based on our discovery that (1) HDAC1 co-purifies with the LTR-binding YY1-LSF repressor complex; (2) the domain of YY1 that interacts with HDAC1 is required to repress the HIV-1 promoter; (3) the expression of HDAC1 augments repression of the LTR by YY1, and (4) the deacetylase inhibitor trichostatin-A blocks repression mediated by YY1. This novel link between HDAC1 recruitment and inhibition of HIV-1 expression by YY1 and LSF, in the natural context of a viral promoter integrated into chromosomal DNA, supports novel HIV therapies described herein and has significant implications for the long-term treatment of AIDS.

### RELATED APPLICATIONS

This application is a continuation application of U.S. application Ser. No. 09/611,949, filed Jul. 6, 2000 (abandoned), which is a continuation-in-part of U.S. application Ser. No. 09/355,010, filed Dec. 8, 1999 (abandoned), which claims the benefit of PCT Application Serial No. PCT/US98/00574, filed Jan. 13, 1998, which claims the benefit of U.S. Provisional Application No. 60/036,242, filed Jan. 23, 1997, the disclosures of each of which are incorporated herein by reference in their entireties.

### ACKNOWLEDGEMENT OF GOVERNMENT SUPPORT

This work was supported in part by National Institutes of Health grant numbers AI 41366 and AI 45297. The United States Government therefore has certain rights in the invention.

### TECHNICAL FIELD OF INVENTION

The present invention relates to our discovery of the molecular mechanism by which a protein complex inhibits HIV-1 transcription. More particularly, the invention relates to a transcription repressor complex or “TRC” containing YY1, LSF, and HDAC1. The components of the TRC cooperate to uniquely bind to at least a portion of the long term repeat (LTR) sequence of HIV-1, preferably the repressor complex sequence (RCS) and inhibit transcription. The invention further relates to pharmaceutical compositions that manipulate this mechanism and methods of use thereof to treat both active and latent HIV infection.

### BACKGROUND OF THE INVENTION

Human immunodeficiency virus is a retrovirus whose major cell target is CD4⁺ T-lymphocytes. HIV-1 infection is mediated through the binding of the virus to the CD4 glycoprotein and other co-receptors. The HIV-1 envelope glycoproteins gp41 and gp120 direct this binding. The gp120 is non-covalently attached to gp41, which is anchored in the viral lipid bilayer. HIV-1 entry is mediated by the high-affinity binding of gp120 to the amino-terminal domain of the CD4 glycoprotein, causing conformational changes in gp120 (McDougal et al., 1986, Science 231:382-385; Helseth et al., 1990, J. Virol. 64:2416-2420; Wain-Hobson, 1996, Nature 384:117-118) and subsequent binding of gp120 to co-receptors, such as CXC-CKR4 and CC-CKR5 (Wu et al., 1996, Nature 384:179; Trkola et al., 1996, Nature 384:184; Wain-Hobson, 1996, Nature 384:117-118).

Studies have shown that the LTR, the promoter of HIV-1, responds to numerous cellular factors (Garcia et al., 1987, EMBO J. 6:3761-3770; Giacca et al., 1992, Virol. 186:133-147; Harrich et al., 1989, J. Virol. 63:2858-2891; Jones, 1989, New Biol. 1:127-135; Jones et al., 1986, Science 232:755-759; Nabel and Baltimore, 1988, Nature 326:711-713; and Wu et al., 1988, J. Virol. 62:218-225). Such factors may augment or repress the production of HIV-1 virions by infected cells.

Two cellular factors, YY1 and LSF that cooperate uniquely in recognition of the region −10 to +27 of the HIV-1 LTR (referred to as the “RCS”, for repressor complex sequence) have previously been identified. These have been shown to specifically and synergistically repress HIV LTR expression and viral production (41, 49). Antibodies to either YY1 or LSF inhibit RCS formation, and mutations within the LTR that eliminate LSF binding and RCS formation ablate repression mediated by YY1 and/or LSF (49).

Yin Yang 1 or “YY1” is a widely distributed 68 kDa multifunctional transcriptional regulator with homology to the GLI-Krüppel family of proteins. YY1 has the ability both to activate and repress gene expression (16, 32, 52, 56). It contains plurality of “zinc fingers” as well as two N-terminal transactivation domains, while the C-terminal domain is required for direct DNA binding and for repression of some promoters (2, 4, 17). This broad spectrum of activity has been attributed to bending of DNA, interactions with other factors, or post-transcriptional modification of YY1 (52). However, activity depends on the promoter context and specific protein-protein interactions that YY1 establishes with other regulatory proteins (23, 32-34, 49, 50, 53, 70-72, 76), and with general transcription factors (5, 60).

“YY1” directly interacts with many viral and cellular nuclear factors (Shi et al., 1991, Cell 67:377-388; Lee et al., 1993, Proc. Natl. Acad. Sci. USA 90:6145-6149; Chiang et al., 1995, Science 267:531-536; Zhou et al., 1995, J. Virol. 69:4323-4330). YY1 has been shown to regulate both viral and lymphocyte promoters (Bauknecht et al., 1992, EMBO J. 11:4607-4617; Flanagan et al., 1992, Mol. Cell Biol. 12:38-44; Park and Atchison, 1991, Proc. Natl. Acad, Sci. USA 88:9804-9808; Seto et alt, 1991, Nature 354:241-245; and Shi et al., 1991, Cell 67:377-388).

YY1 (also known as 6, NF-E1, UCRBP or CF1) has been shown to cooperate uniquely in recognition of the region of the HIV-1 LTR referred to as the repressor complex sequence or RCS (Malim et al., 1989, J. Virol. 63:3213-9; Shi et al., 1997, Biochim Biophys Acta. 1332:F49-66; Shrivastava, et al., 1994, Nucleic Acids Res. 22:5151-5; Wu, et al., 1988, EMBO J. 7:2117-30). YY1 has previously been shown to repress HIV-1 transcription and virion production (Margolis, et al., 1994, J. Virol. 68:905-910). Moreover YY1 had been found to inhibit HIV-1 LTR transcription in vivo (Margolis et alt 1994, J. Virol. 68:905-910).

LSF is a lymphoid transcription factor that has been shown to repress LTR transcription in in vitro, but not in in vivo assays (Kato et al., 1991, Science 251:1476-1479; Yoon et al., 1994, Mol. Cell. Biol. 14:1776-1785). LSF is the predominantly expressed member of a family of proteins (also termed LBF-1, CP-2, LBP-1a, b, c and d) that are produced from the differential splicing from two related genes (55, 73). All bind DNA except for LSF-ID (LBP1-d), which lacks a central encoding exon. LSF (LBF-1, CP-2) recognizes the same LTR sequence as YY1 (Huan et al., 1990, Genes Dev, 4:287-298; Lim et al., 1992, Mol. Cell. Bio. 12:828-835; Garcia et al., 1987, EMBO J. 6:3761-3770; Kato et al., 1991, Science 251:1476-1479; Yoon et al., 1994, Mol. Cell. Biol. 14:1776-1785). LSF binding to the LTR is associated with direct repression of transcription in vitro (18, 29, 44). However, transient expression of LSF alone had no observable effect on expression from the HIV LTR (49, 73, 75).

As we reported in parent application Ser. No. 09/355,010 (International Publication No. WO 98/33067), the entire contents of which are incorporated by reference herein, YY1 and LSF form a complex (the TRC) that binds the HIV LTR, particularly the RCS site, and blocks transcription. As discussed in detail herein, we have discovered that the complex further contains HDAC1. A molecular mechanism of TRC-associated repression of HIV-1 which is also described herein. The invention is based on the discovery that (1) HDAC1 copurifies with the LTR-binding YY1-LSF repressor complex; (2) the domain of YY1 that interacts with HDAC1 is required to repress the HIV-1 promoter; (3) the expression of HDAC1 augments repression of the LTR by YY1, and (4) the deacetylase inhibitor trichostatin-A blocks repression mediated by YY1. This novel link between HDAC1 recruitment and inhibition of HIV-1 expression by YY1 and LSF, in the natural context of a viral promoter integrated into chromosomal DNA, supports novel HIV therapies described herein and has significant implications for the long-term treatment of AIDS.

A subpopulation of stably infected CD4⁺ T lymphocytes containing integrated proviral DNA capable of producing virus upon stimulation has been identified in HIV⁺ individuals (6, 7, 8, 15, 68). As antiretroviral therapy now allows significant inhibition of active HIV-1 replication, an understanding of factors that establish or maintain the integrated proviral state takes on new relevance. Potent repression of LTR transcription could allow an activated, infected cell to return to the resting state and establish stable nonproductive infection. This may occur via changes in local chromatin architecture surrounding the HIV promoter. While activation of the HIV LTR has been shown to be associated with changes in chromatin structure (13, 46, 51, 61-63), factors that result in durable repression of LTR expression are less well known.

In view of the lack of methods of down-regulating transcription of the HIV-1 LTR, it is clear that there exists in the art a need for effective therapies that regulate transcriptional repression of the HIV-1 LTR. The present invention solves this problem by providing methods for the improvement of HIV-1 LTR transcriptional repression. It also solves this problem by providing methods for improved antagonism of LTR transcriptional repression, thereby leading to conditions in which HIV cannot establish a virologically latent intracellular infection, and that will allow for clearance of HIV infection when used in combination with other potent anti-viral agents. The regulation of proviral expression within this reservoir of infected CD4⁺ cells may take on new relevance as potent combination antiretroviral therapies allow the depletion of HIV from productively infected cell populations.

### SUMMARY OF THE INVENTION

The present invention relates to our discovery of the molecular mechanism by which the YY1/LSF repressor complex inhibits HIV-1 transcription. Our discovery of a novel link between HDAC1 recruitment and inhibition of HIV-1 expression by YY1 and LSF, in the natural context of a viral promoter integrated into chromosomal DNA, supports novel HIV therapies described herein and has significant implications for the long-term treatment of AIDS.

An object of the present invention is to provide a method of repressing HIV transcription, thereby treating or preventing HIV infection in a human subject in need of such treatment.

In one preferred embodiment, this method comprises administering to the subject an effective amount of a preparation of a transcription repressor complex comprising YY1 (or a derivative or analog thereof), LSF (or a derivative or analog thereof), and HDAC1 (or a derivative or analog thereof). Preferred derivatives and analogs are discussed in detail herein.

In another preferred embodiment, this method comprises administering to the subject an effective amount of a preparation comprising an agent that enhances the binding of a YY1 (or a derivative or analog thereof) to HDAC1 (or a derivative or analog thereof).

In another preferred embodiment, this method comprises administering to the subject an effective amount of an agent that enhances recruitment of HDAC1 (or a derivative or analog thereof by YY1 (or a derivative or analog thereof to the LTR RCS site.

In another preferred embodiment, this method comprises administering to the subject an effective amount of a preparation comprising an agent that enhances the activity of HDAC1, or a derivative or analog thereof.

In another preferred embodiment, this method comprises administering to the subject an effective amount of an agent that enhances the expression of HDAC1, or a derivative or analog thereof.

In another preferred embodiment, this method comprises administering to the subject an effective amount of an agent that up-regulates the expression of HDAC1, or a derivative or analog thereof.

In another preferred embodiment, this method comprises administering to the subject an effective amount of a nucleic acid or combination of nucleic acids comprising: one or more nucleotide sequences encoding YY1 (or a derivative or analog thereof); one or more nucleotide sequences encoding LSF (or a derivative or analog thereof); and one or more nucleotide sequences encoding HDAC1 (or a derivative or analog thereof).

A further object of the present invention is to provide a method of treating quiescent reservoirs of HIV infection in a human subject in need of such treatment comprising the steps of:
(a) administering to the subject an amount of an agent that down-regulates the expression of HDAC1, or a derivative or analog thereof, the amount effective to down-regulate TRC-associated repression of HIV transcription;
(b) allowing latent, quiescent reservoirs of HIV to become actively transcribing; and
(c) treating the subject with an effective amount of an antiretroviral agent.

In an alternate embodiment, this method comprises the administration of an agent that inhibits the expression of HDAC1 or a derivative or analog thereof, the amount being effective to inhibit TRC-associated repression of HIV transcription.

Another object of the present invention is to provide a composition comprising the TRC complex of YY1, LSF and HDAC1 and a method of using this composition to screen for analogs thereof, the analogs having anti-HIV activity or HIV transcription repressing activity.

A further object of the present invention is to provide a pharmaceutical composition that represses TRC-associated HIV transcription, thereby providing a novel therapy for HIV infection.

In a preferred embodiment, this pharmaceutical composition comprises an effective amount of YY1, LSF and HDAC1, or derivatives or analogs thereof.

A further object of the present invention is to provide a pharmaceutical composition comprising an effective amount of one or more anti-HIV agent, said agent repressing TRC-associated HIV transcription, thereby providing a novel therapy for HIV infection.

In another preferred embodiment, this pharmaceutical composition comprises an effective amount of a nucleic acid or combination of nucleic acids comprising: one or more nucleotide sequences encoding YY1 (or a derivative or analog thereof; one or more nucleotide sequences encoding LSF (or a derivative or analog thereof); and one or more nucleotide sequences encoding HDAC1 (or a derivative or analog thereof).

In another preferred embodiment, this pharmaceutical composition comprises an effective amount of an agent that enhances the binding of a YY1 or a derivative or analog thereof to HDAC1 or a derivative or analog thereof.

In another preferred embodiment, this pharmaceutical composition comprises an effective amount of an agent that enhances recruitment of HDAC1, or a derivative or analog thereof, by YY1, or a derivative or analog thereof, to the HIV LTR RCS site.

In another preferred embodiment, this pharmaceutical composition comprises an effective amount of an agent that enhances the activity of HDAC1, or a derivative or analog thereof, said amount effective to repress HIV transcription.

In another preferred embodiment, this pharmaceutical composition comprises an effective amount of an agent that enhances the expression of HDAC1 or a derivative or analog thereof.

In another preferred embodiment, this pharmaceutical composition comprises an effective amount of an agent that up-regulates the expression of HDAC1 or a derivative or analog thereof.

In another preferred embodiment, this pharmaceutical composition comprises an effective amount of YY1, LSF and HDAC1.

Yet another object of the present invention is to provide methods of identifying, screening, and/or isolating compounds having activities that affect or regulate the TRC complex and its repression of HIV LTR-driven transcription.

A further object of the invention is to provide methods of identifying, screening and/or isolating compounds with “anti-HIV activity”. Such compounds find therapeutic utility and may be used to form pharmaceutical compositions for the treatment of active and/or latent HIV infections. Of particular interest are compounds that affect or regulate HDAC1 expression or activity or affect or regulate the HDAC1-recruiting activity of YY1.

In one preferred embodiment, the compounds of interest act to enhance, augment or up-regulate the expression of HDAC1. Such compounds find therapeutic utility in the treatment of active HIV infections.

In another preferred embodiment, the compounds of interest act to enhance or augment the activity of HDAC1. Such compounds find utility in the therapeutic treatment of active HIV infections.

In another preferred embodiment, the compounds of interest act to enhance or augment the ability of YY1 to recruit HDAC1 to the RCS site of the HIV LTR. Such compounds find therapeutic utility in the treatment of active HIV infections.

In an alternate embodiment, the compounds of interest act to inhibit, repress or down-regulate the expression of HDAC1. Such compounds find therapeutic utility in the treatment of latent HIV infections.

In another embodiment, the compounds of interest act to inhibit or repress the activity of HDAC1. Such compounds find therapeutic utility in the treatment of active HIV infections.

In another preferred embodiment, the compounds of interest act to inhibit or repress the ability of YY1 to recruit HDAC1 to the RCS site of the HIV LTR. Such compounds find therapeutic utility in the treatment of active HIV infections.

A further object of the present invention is to provide a method and composition for modulating the histone structure of the RCS site of the HIV LTR so as to modulate the activity of the TRC complex and TRC-associated repression of HIV transcription.

Yet another object of the present invention is to provide a method and composition for modulating the association between HDAC1 and YY1 so as to modulate the activity of the TRC complex and TRC-associated repression of HIV transcription.

These and other objects, aspects, features, and advantages of the invention will become evident upon reference to the following description.

### BRIEF DESCRIPTION OF THE DRAWINGS

FIGS. 1A-1C depicting the association between YY1 and LSF in vivo and in vitro, in the absence of a DNA binding site or other factors.

FIG. 1A is a western blot depicting the immunoprecipitations of Jurkat nuclear extracts using either α-YY1, α-LSF or a nonspecific rabbit polyclonal antiserum. Mock immunoprecipitations were performed in the absence of antibody. Precipitates were assayed by Western blot using α-LSF. Approximately 75% of the LSF protein recovered by α-LSF is also immunoprecipitated by α-YY1. To demonstrate the recognition of YY1, a western blot of input nuclear extract is displayed at the right.

FIG. 1B depicts the EMSA results performed using the RCS binding site and the indicated amounts of LSF and YY1; total amount of protein was normalized by the addition of BSA. The mobility of the native RCS complex formed by nuclear extract is displayed at the right.

FIG. 1C depicts the EMSA results performed using the RCS binding site and the indicated amounts of either α-YY1 or α-LSF. Addition of α-YY1 had no effect on the LSF complexes in the absence of YY1 protein (not shown).

FIGS. 2A-2C map the YY1/LSF interaction domains.

FIG. 2A is a representation of LSF deletion mutants used to identify the region of interaction between LSF with YY1. The terminology is defined as follows: ΔA represents a deletion up to codon A, BΔ represents a deletion after codon B, AΔB represents a deletion between codons A and B, XX represents mutated single codons. The amount of LSF bound to GST-YY1 varied from 2.5 to 7% of the input depending on the experiment. All values were normalized to the amount of wild-type LSF bound to GST-YY1 within the experiment.

FIG. 2B is a representative autoradiograph showing input LSF constructs, LSF constructs retained by GST-YY1 and by GST, respectively.

FIG. 2C is a graphical representation of the YY1 chimeras, all of which contained the GST tag. All constructs also contained the N-terminal region of YY1 (amino acids 1-294) except YZrns, which lacked this region. YY1 is the full-length wild type YY1 molecule. Non-shaded regions represent GFI-1 zinc fingers (a related Krüppel zinc finger protein). Y/GFI contains only GFI-1 zinc fingers, Chi 1 contained the first YY1 zinc finger, Chi 2 the first two YY1 zinc fingers, Chi 5 the last two YY1 zinc fingers, Chi 7 the second YY1 zinc finger only, and ZnFs all four YY1 zinc fingers without the YY1 amino-terminal region. The first two zinc fingers of YY1 are required for optimal binding of LSF. Chi 1, Chi 2, Chi 7, and YY1 bound LSF whereas Chi 5, GFI-1 and GST exhibited background levels of LSF binding. A lane containing only a diluted aliquot of labeled LSF serves as a marker. When normalized for protein concentration, a YZnFs construct expressing only the YY1 zinc fingers fused to GST binds LSF with equal avidity to intact GST-YY1. Background levels of binding varied between experiments, as shown.

FIG. 3 shows that repression by YY1 and LSF require functional LSF and HDAC1 interaction competent YY1. Expression of an integrated LTR-CAT reporter in HeLa-CD4-LTR cells, when activated by 200 ng of pAR-Tat, is inhibited by 2.5 μg CMV-YY1 or 2.5 μg of both CMV-YY1 and CMV-LSF. 2.5 μg CMV-LSF had no effect on expression of CAT. 2.5 μg of the dominant negative form of LSF (pCMV-LSF 234QL/236KE), incapable of binding DNA but capable of forming inactive multimers, blocks inhibition of Tat-activated LTR expression by 2.5 μg of YY1. 2.5 μg CMV-YY1Δ154-199, incapable of interacting with HDAC1, was unable to inhibit Tat-activated expression. All transfections received a total of 5 μg CMV promoter-driven plasmid. Data from at least four independent transfections, normalized for expression of cotransfected β-actin-luciferase.

FIGS. 4A and 4B show that YY1, LSF and HDAC1 copurify with RCS-binding activity.

FIG. 4A shows the activities of crude nuclear extract and elution fractions from an RCS DNA-affinity chromatography column. EMSA (top panel) using the RCS probe. Western blot using rabbit polyclonal αYY1 (second panel), rabbit polyclonal α-CP2 (LSF) antiserum (third panel), and rabbit polyclonal α-HDAC1/2 antibody bottom panel). EMSA were performed with 4 μg of nuclear extract and 20 ng of DNA-affinity column eluate. Western blot was performed with 20 μg of nuclear extract and 200 ng of DNA-affinity column eluate. Arrow indicates YY1 specific complex, as validated by α-YY1 interference in EMSA. Molecular weight markers are indicated.

FIG. 41B demonstrates that the HDAC1 activity of DNA affinity chromatography fractions correlates with the presence of the YY1/LSF complex.

FIG. 5 is a graph depicting the affect of YY1 on the production of HIV in vitro. Production of HIV-1 is inhibited by YY1 but not by YY1Δ154-199 lacking the HDAC1 interaction domain following transfection of HeLa cells with 0.5 μg of the CXCR4 prototypic clone pNL4-3 (left panel), or 1 μg of the CCR5 prototypic clone pYU-2 (right panel). Data is representative of three transfections.

FIG. 6 is a model of recruitment by LSF of YY1, and then HDAC1 to the HIV promoter.

FIG. 7 is the HIV-1 LTR partial nucleotide sequence (SEQ ID NO: 1). Note that the transcription start site is nucleotide +1 (the “g” indicated by arrow); the nucleotides upstream the transcription start site have a negative numeration, while the nucleotides downstream the transcription start site have a positive numeration. The two NF-KB binding sites, the three Sp1 binding sites and the Repressor Complex Sequence (RCS) are labeled.

FIG. 8 is the human YY1 cDNA nucleotide sequence (SEQ ID NO: 2) and amino acid sequence (SEQ ID NO: 3)

FIG. 9 is the human LSF cDNA nucleotide sequence (SEQ ID NO: 4) and amino acid sequence (SEQ ID NO: 5)

FIG. 10A is the human histone deacetylase 1 amino acid sequence (SEQ ID NO: 6)

FIG. 10B is the human histone deacetylase 1 mRNA nucleotide sequence (SEQ ID NO: 7)

### DETAILED DESCRIPTION OF THE PREFERRED EMBODIMENTS

Genetic and biochemical studies have established that chromatin in living cells critically affects transcriptional competence of a promoter sequence (3, 14, 36, 58, 67). A number of recent reports have documented the importance of histone deacetylases as the effector molecules of transcriptional down-regulation in many genes (11, 20, 25, 39, 47). In addition, several transcriptional repressors have been described that tether HDACs to the promoter (2, 3, 21, 28, 31, 42, 43, 71, 72, 74).

We mapped the interactions of LSF and YY1 using a number of chimeric YY1 and truncated LSF constructs to determine the domains that participate in complex formation and regulation of the HIV promoter. We subsequently discovered a novel molecular mechanism of repression of an integrated HIV provirus in vivo, wherein LSF is required for recruitment of YY1 to the HIV LTR, and repression is mediated by YY1 via the action of HDAC1.

We discovered that the YY1/LSF complex copurifies with HDAC1, identified by both Western blot analysis and enzymatic activity assay. Deletion of a glycine/alanine-rich domain of YY1, previously shown to specifically direct the interaction between YY1 and HDAC1 (71), ablates the ability of YY1 to repress the HIV-1 LTR. Further YY1-mediated repression of the LTR is ablated by the deacetylase inhibitor trichostatin A. This is the first discovery of the presence of HDAC1 in the transcription repressor complex. This is also the first discovery of a molecular mechanism by which the TRC represses HIV-1 transcription.

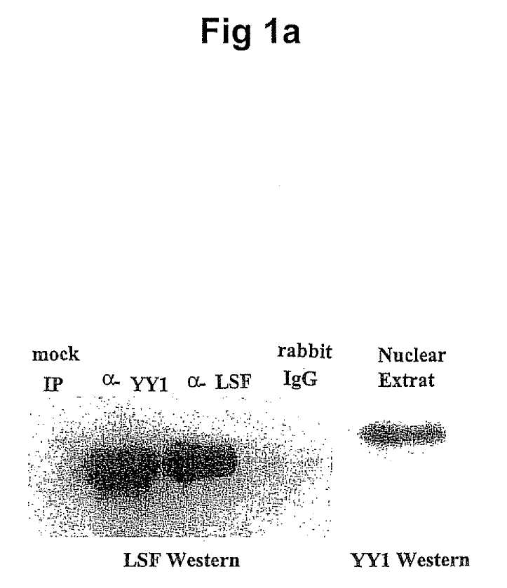

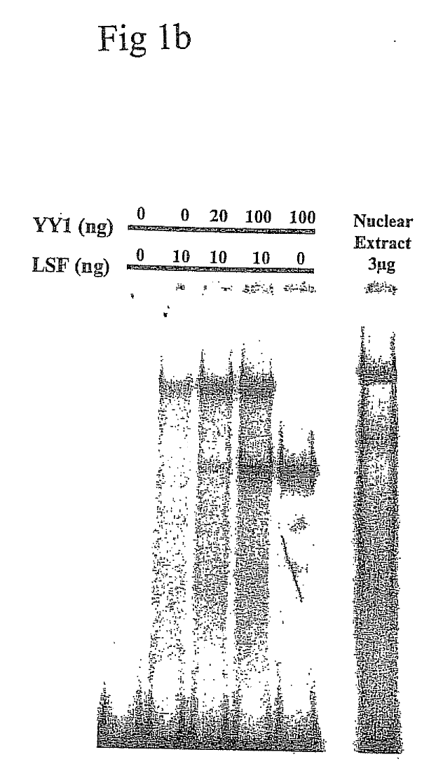

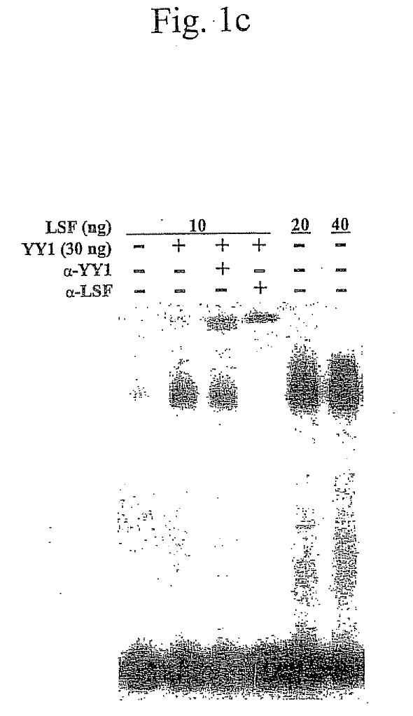

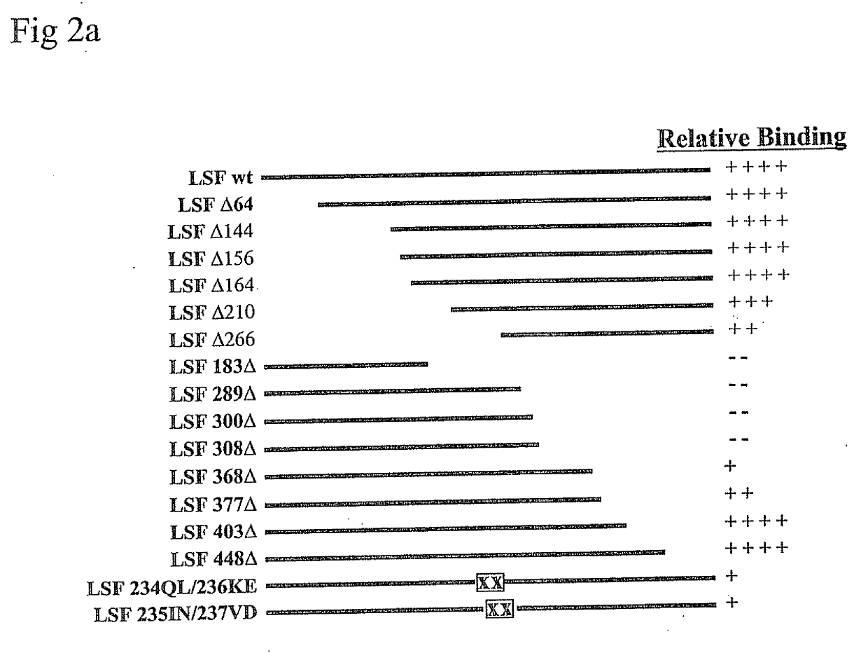

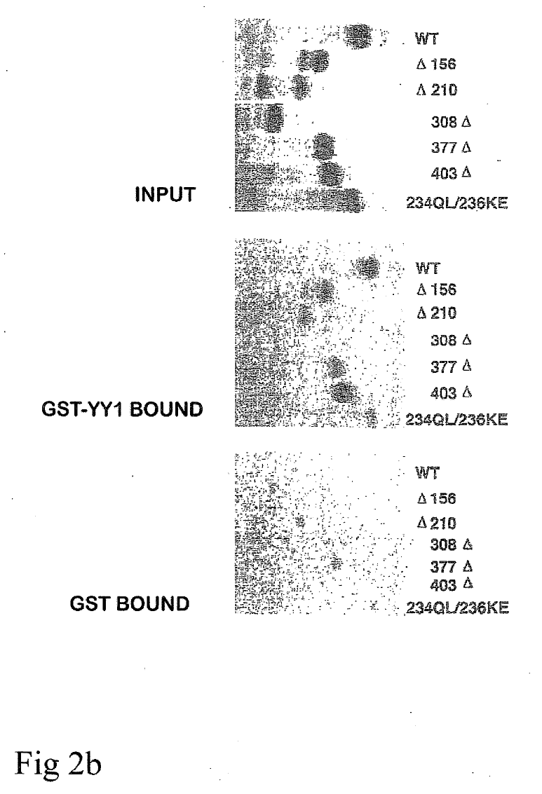

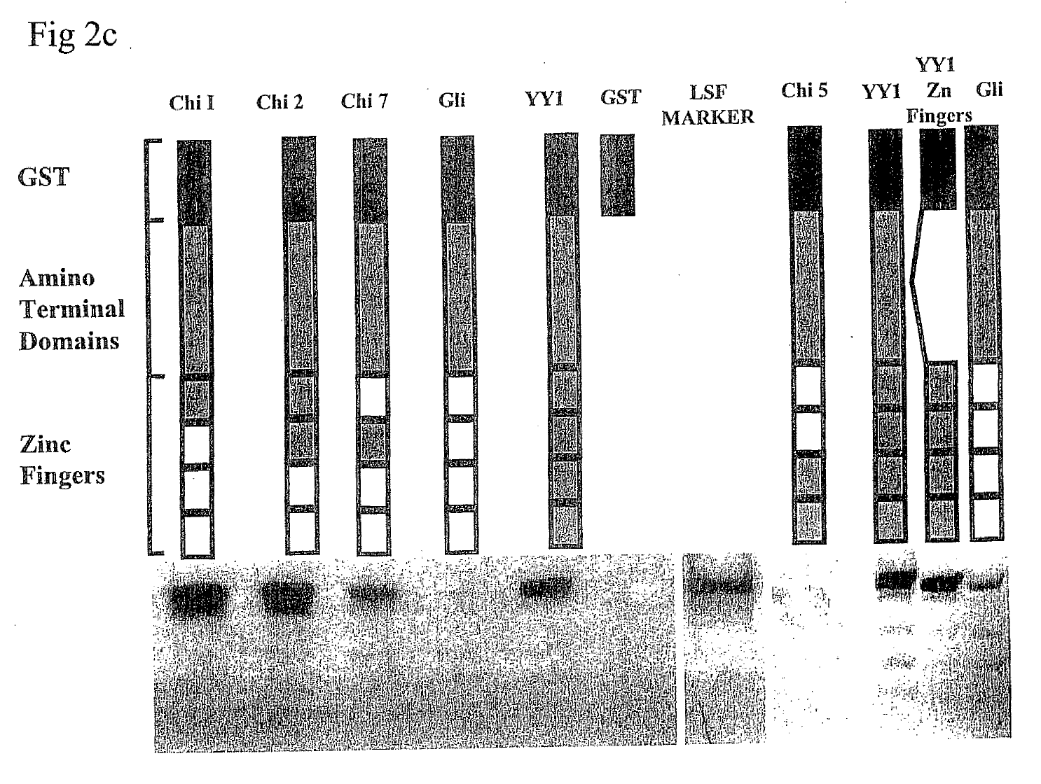

As discussed herein, particularly in the Examples section, we show that LSF and YY1 interact with one another both in vitro and in vivo. Interaction was observed in the absence of: (a) DNA binding site, (b) other cellular factors, and (c) YY1 C-terminal zinc fingers required for DNA binding to canonical YY1 sites (FIGS. 1, 2, and ref 17). As a majority of the LSF that can be recovered by immunoprecipitation can also be recovered in association with YY1, this complex is likely to be preformed in the cell prior to binding to viral regulatory elements. Further evidence of YY1 and LSF interaction is provided by the observation that YY1 alone does not bind the RCS site in EMSA, but α-YY1 supershifted a significant fraction of the RCS-protein complex.

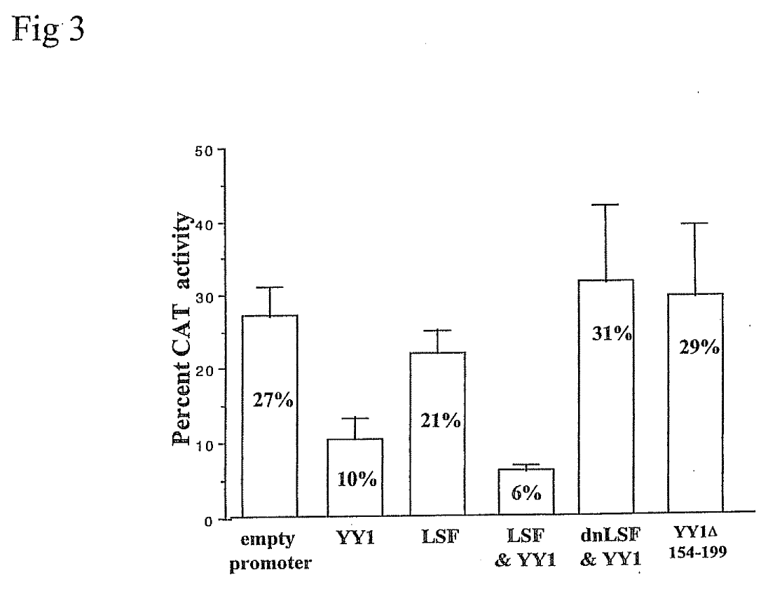

Overexpression of LSF alone does not repress transcription from the HIV LTR (49, 75). However, an LTR reporter gene is inhibited by YY1 expression, and this effect is augmented by coexpression of LSF. This occurs in the context of both plasmid based (49) and chromatin based reporter genes (FIG. 3). Further, both YY1 and LSF function are required for inhibition of the HIV LTR. While expression of LSF does not significantly inhibit LTR expression, LSF synergizes with YY1 in repression, and dominant negative LSF prevents repression by YY1 (FIG. 3). Consistent with this model, preliminary studies show that the replication of a provirus containing TAR mutations that block RCS formation, but allow Tat function (48) is unaffected by YY1 (data not shown).

YY1 is known to interact with a number of cellular factors via a Gly/Ala-rich region within residues 154-198 (2). LSF, however, interacts with the zinc finger domain of YY1. The Gly/Ala domain was present on all chimeric YY1 constructs, but only YY1, chimeras 1, 2 and 7, and the YY1 zinc fingers alone were able to specifically bind LSF in vitro (FIG. 2c). These results indicate that the first and to a lesser extent the second zinc finger domain of YY1 participate in interaction with LSF. While zinc fingers often mediate DNA binding, examples of protein-protein interactions mediated by zinc fingers have been documented (3, 19, 30, 65).

Our findings show that LSF is required for recruitment of YY1 to the HIV promoter. LSF appears to facilitate YY1 recognition of the LTR, guiding YY1 onto a site that was inaccessible or of low affinity in its absence. However, YY1 may enter the RCS complex solely through protein-protein interaction with LSF. Given the sensitivity of this interaction to mutations within the core of LSF that impair multimerization, it is likely that YY1 recognizes a structure displayed by LSF multimers. The location of the RCS within the HIV LTR may position YY1 to directly inhibit the basal transcription complex or activators of the LTR, as well as recruit mediators of repression such as HDACs.

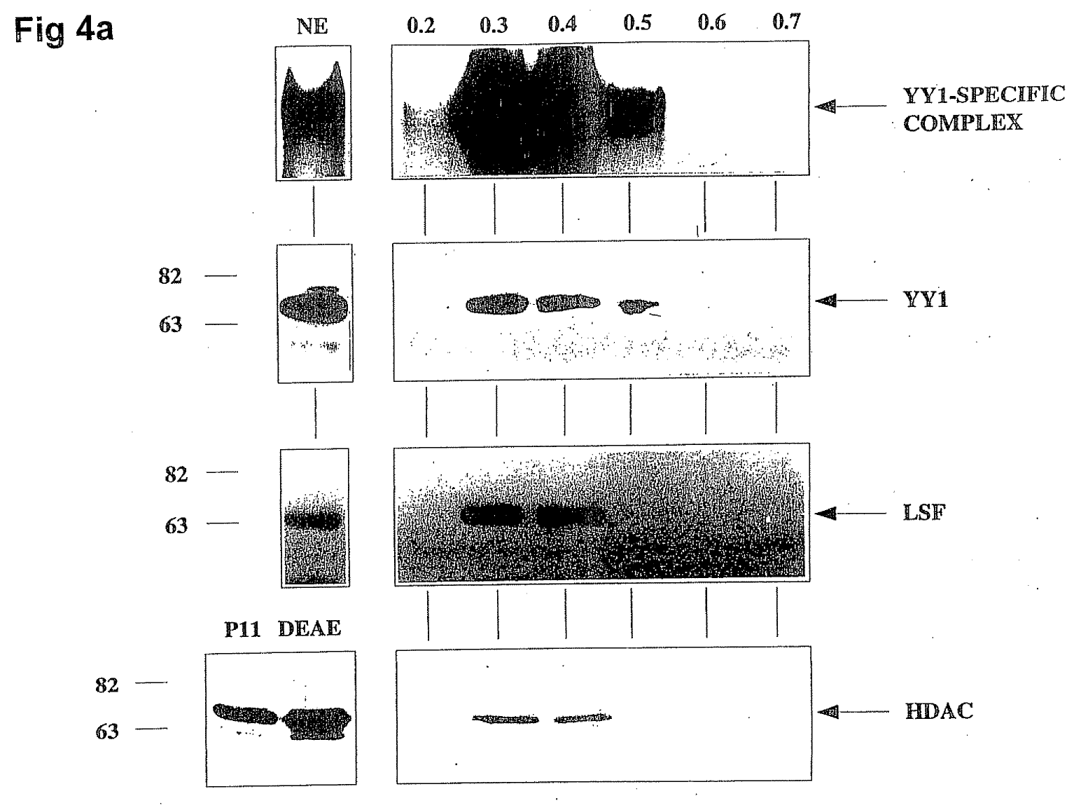

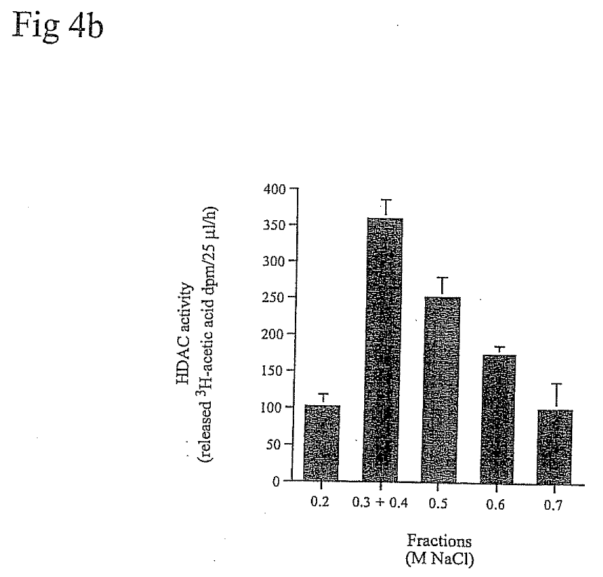

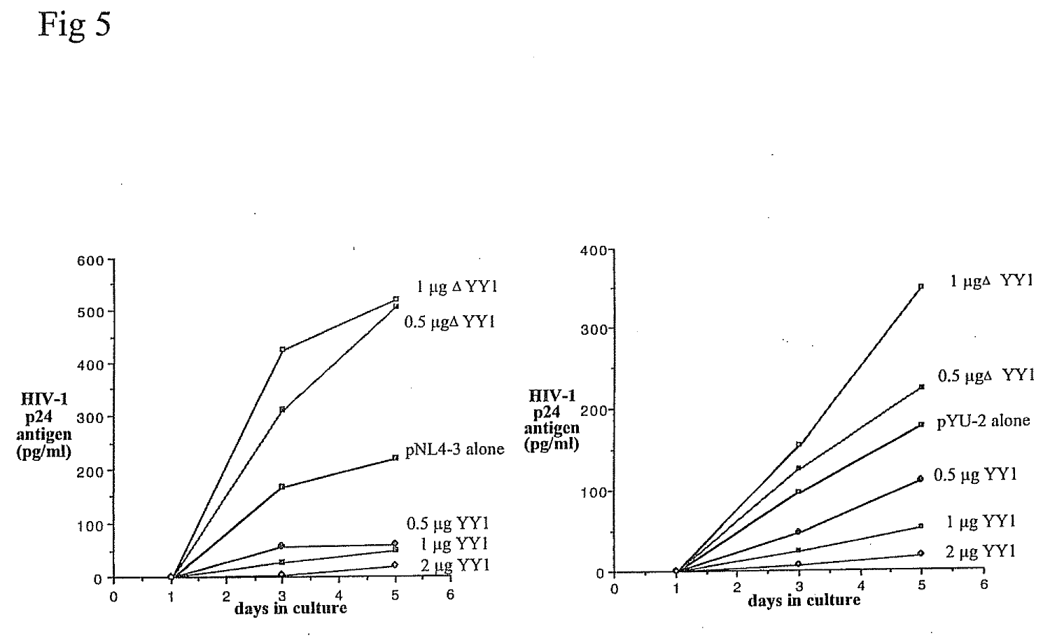

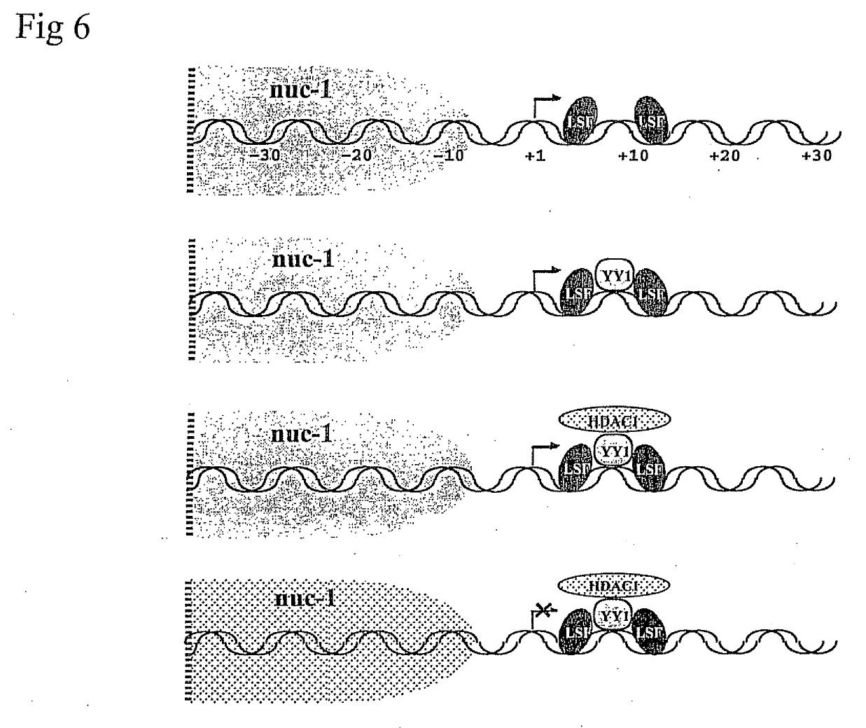

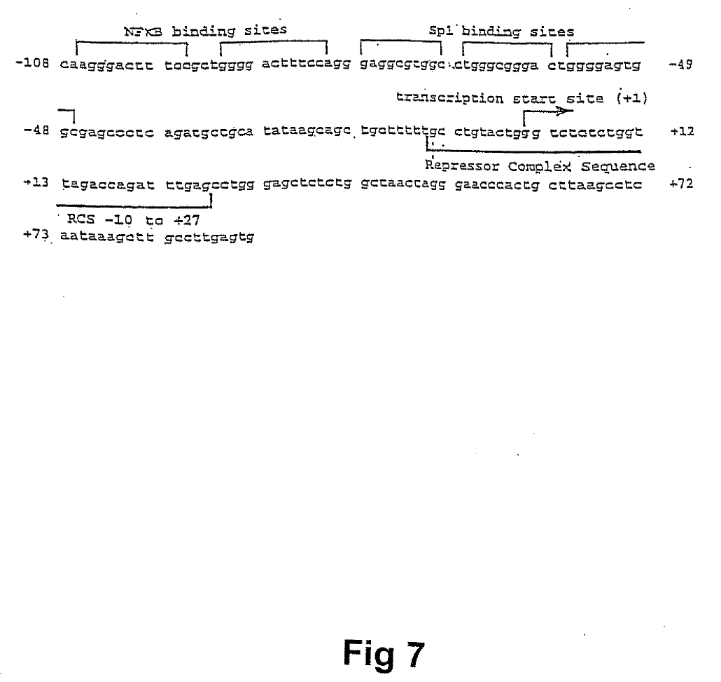

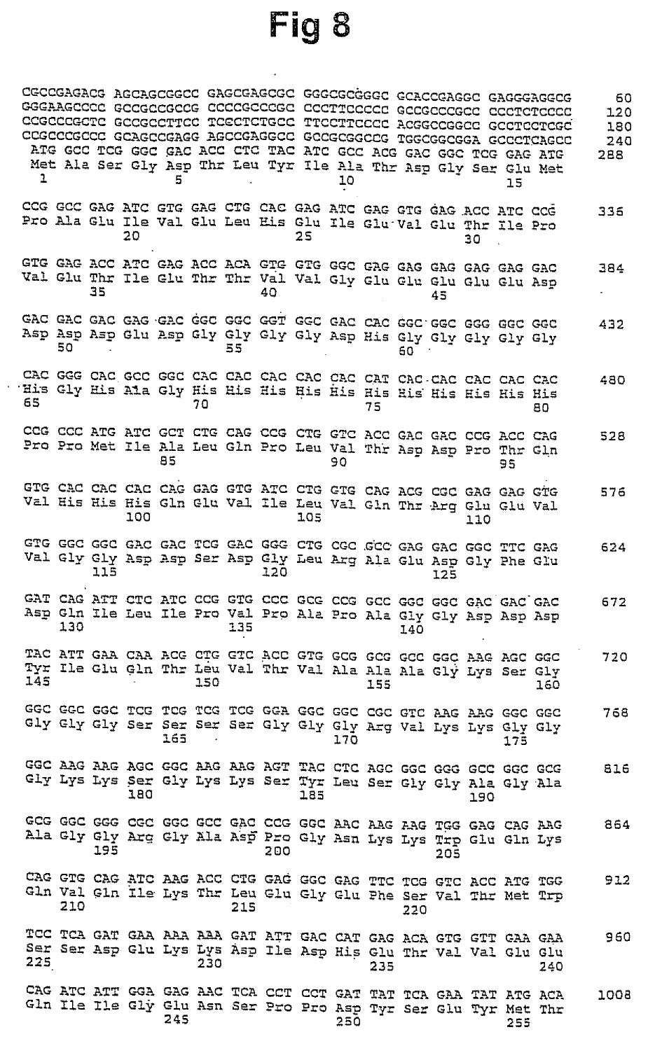

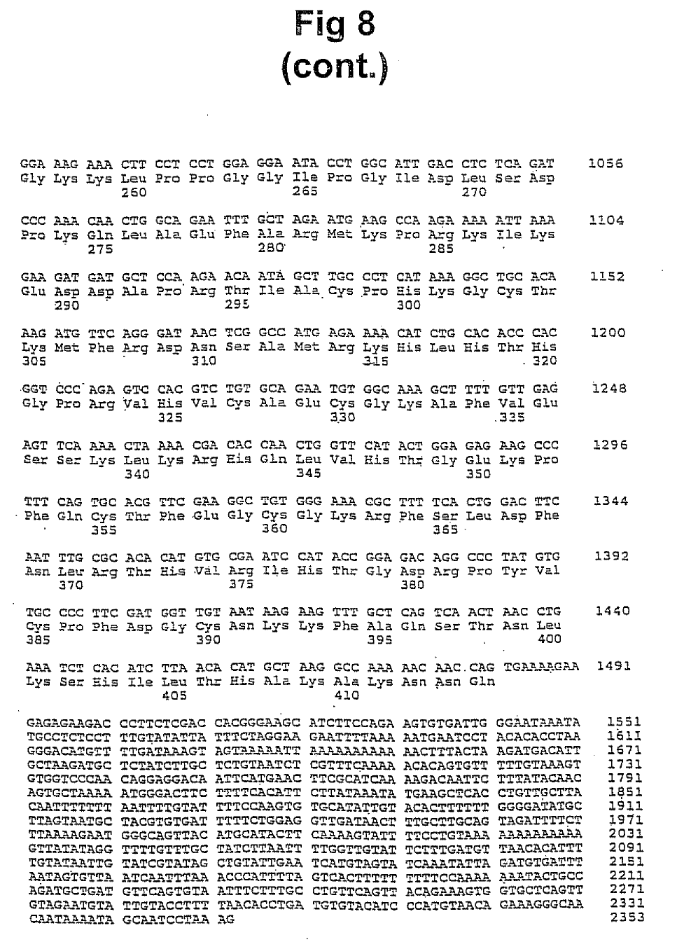

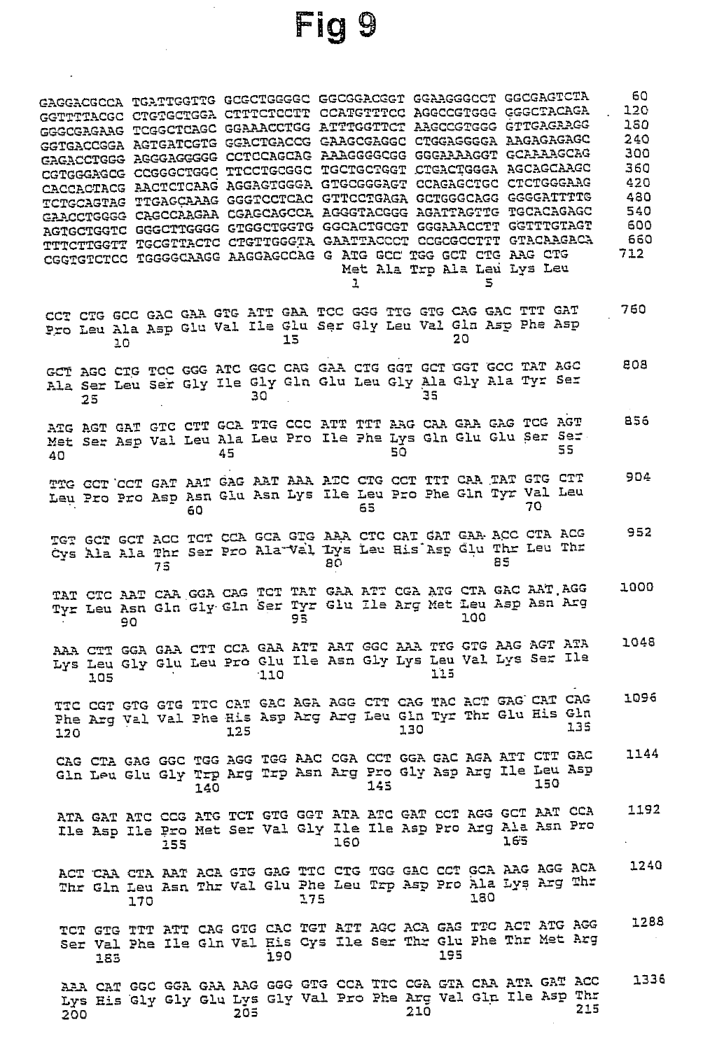

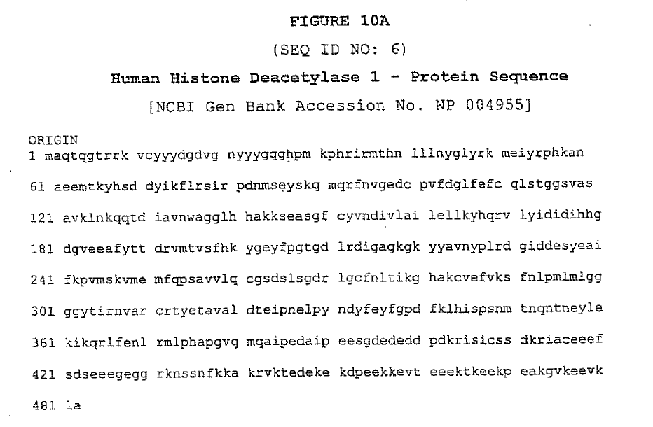

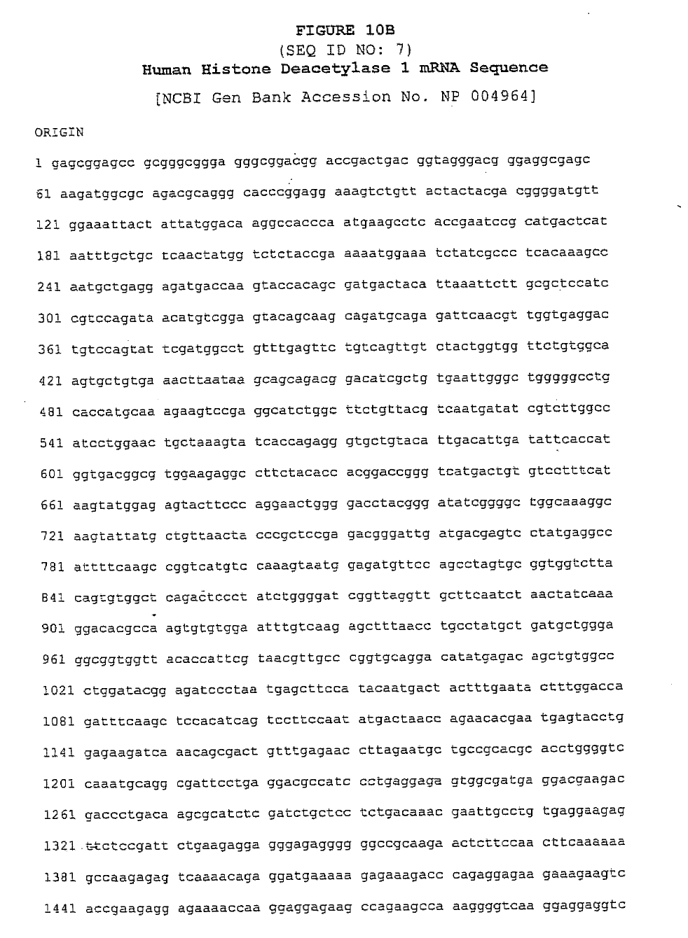

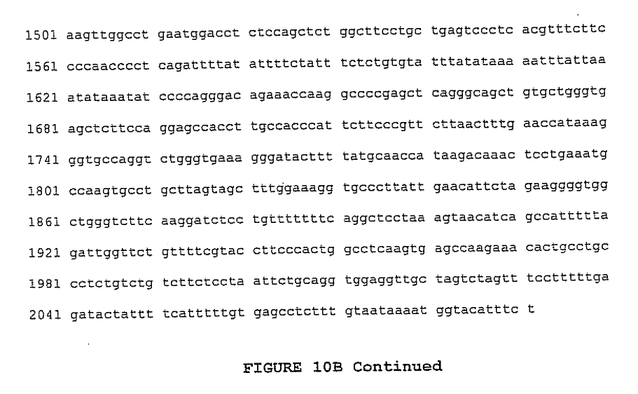

The multiprotein repressor complex containing the human factors YY1 and LSF, previously isolated from CEM cells, detected in primary T cells, and shown to repress the HIV-1 LTR, copurifies with a 65-kDa protein which we have identified as HDAC1 (FIG. 4a).

The anti-HDAC1 antibody used to perform the Western blot analysis was reactive to both is HDAC1 and HDAC2, but not to HDAC3 (72). Molecular weight found in western blot analysis (59, 71) suggest that HDAC1 is the protein copurified with YY1 and LSF.

We have demonstrated that the Gly/Ala-rich domain of YY1, which mediates the interaction with HDAC1 (71), is required for efficient repression of the HIVE LTR by the YY1-LSF complex (FIG. 3). This suggests that recruitment of a histone deacetylase is a necessary event in the mechanism of repression of HIV-1 gene expression by YY1. Indeed, the fact that transfection of YY1Δ154-199 resulted in modest up-regulation of LTR expression and HIV production suggests that LTR expression may in part reflect the competing influences of cellular HDAC1 and histone acetyltransferase activity.

Studies of the chromatin structure of the integrated HIV-1 provirus in chronically and acutely infected cells lines have detected the presence of a large nucleosome-free, DNase I-hypersensitive region spanning nucleotides 223 to 450 of the HIV-1 genome. This corresponds to the portion of the LTR including the enhancer and the promoter regions, up to the transcription start site. Upon treatment with TPA or TNF-α, the 3′ boundary of the nucleosome-free region was extended a further 140 nucleotides, indicating the alteration of the nucleosome, termed nuc-1 (51, 61-63). Additional DNase I-hypersensitive sites and nucleosome-protected regions have been identified all along the integrated HIV-1 genome (61, 62).

More recently Pazin et al (46) have shown that binding of both Sp1 and the p50 subunit of NF-KB to the HIV-1 LTR alters the local nucleosomal array in vicinity of the HIV-1 promoter and produces the DNase I-hypersensitive region between nucleotides 223 and 450. However, it is the p65 subunit of NF-KB that induces changes in the nucleosome nuc-1, perhaps through the recruitment of a histone acetyltransferase (51), and enhances transcriptional activity (46).

Our previous results suggested that LSF allowed YY1 to recognize a site on the LTR that YY1 could not bind by itself (49). Therefore, LSF might primarily act as a docking molecule for YY1, which in turn acts by tethering histone deacetylase (FIG. 6). In this model YY1 may be a limiting factor for repression of the LTR, required for the recruitment of the histone deacetylase to the HIV-1 promoter. The finding that overexpression of the mutant YY1Δ154-199 results in activation of HIV expression is consistent with such a model. El Kharroubi et al (13) has shown that activation of the integrated proviral HIV genome requires alteration of the local chromatin via acetylation of the nucleosome adjacent to the start site. The recruitment of HDAC1 by YY1 might prevent such changes in the local chromatin, maintaining the nucleosome in a deacetylated state, preserving higher order nucleosome structure and thereby inhibiting gene expression (FIG. 6).

In vitro assays have shown that assembly of nuc-1 on naked HIV-1 DNA can be inhibited by the presence of LSF (51). Further, one previous report suggested that LSF is important for efficient activation of the HIV-1 LTR (26), although this has been disputed (75). However, we find no evidence that wild type LSF activates LTR expression. While previous data that LSF is an activator of HIV conflicts with our findings, these studies were performed in very different experimental systems. Indeed, through interaction with other factors, in the absence of YY1, or in other cellular milieus LSF might direct LTR activation.

The HIV-1 enhancer and promoter possess a multiplicity of sequences recognized by cellular and viral regulatory factors. The role of cellular enhancers such as Sp1 and NF-KB, and the viral activator Tat in active HIV gene expression has been extensively studied. As discussed above, changes in chromatin structure about an integrated HIV promoter during activation have been documented. However, mechanisms that down-regulate HIV expression are largely unknown. We have shown that YY1 and LSF are capable of cooperating to inhibit HIV transcription. Of the many possible mechanisms through which YY1 might down-regulate transcription, we can now link this function to the recruitment of a histone deacetylase; our studies strongly suggest this enzyme is histone deacetylase 1. Thus the YY1-LSF repressor complex recruits factors capable of potent and durable inhibition of HIV-1 LTR promoter expression.

A large nucleosome-free, DNase I-hypersensitive region spanning nucleotides 223 to 450 of the HIV-1 has been observed in the chromatin structure of the integrated HIV-1 provirus in chronically and acutely infected cells lines. Activation of LTR expression extends the 3′ boundary of this nucleosome-free region a further 140 nucleotides. Each nucleosome is entwined by 1.65 turns of a left-handed superhelix of DNA that corresponds to 147 basepair. This indicates that the DNA protected by one nucleosome has been exposed, presumably by remodeling of the nucleosome structure. The binding of both Sp1 and the p50 or p65 subunits of NF-KB to the HIV-1 LTR alters the local nucleosomal array in vicinity of the HIV-1 promoter, and perhaps through the recruitment of a histone acetyltransferase enhances transcriptional activity. El Kharroubi et al (13) have also shown that activation of the integrated proviral HIV genome requires alteration of the local chromatin via acetylation of the nucleosome adjacent to the start site. Our findings imply that recruitment of HDAC1 by YY1 might prevent such changes in the local chromatin, maintaining the nucleosome in a deacetylated state and inhibiting HIV expression.

We propose a dynamic model of HIV LTR regulation that would allow the establishment of virological latency in rare CD4 T cells. Following T cell activation necessary for viral entry, reverse transcription, and other steps of the viral life cycle which lead to proviral integration, the rare activated cell avoids apoptosis or viral or immune-mediated destruction. Dampening of LTR expression by YY1 and LSF may play an important role at this stage. This cell then follows pathways that typically reestablish the resting, memory state. The HIV-1 LTR remains silent due to the predominant effects of repressor molecules, resulting in an inaccessible chromatin structure about the LTR. This cell may later exit virological latency if it encounters appropriate stimuli that to increase nuclear levels of NF-KB, again changing LTR chromatin structure. Levels of the viral activator tat then increase within the cell, driving the equilibrium towards viral expression.

### DEFINITIONS

As used herein, the “HIV LTR” refers to the long term repeat sequence on the HIV that acts as the primary promoter of HIV transcription. The repressor complex sequence or “RCS” refers to the region −10 to +27 of the HIV-1 LTR. The HIV LTR sequence is set forth in FIG. 8 (SEQ ID NO: 1).

As used herein, the transcription repressor complex or “TRC” refers to a protein complex containing YY1, LSF, and HDAC1 that binds to the LTR RCS and inhibits HIV transcription.

As used herein, Yin Yang 1 or “YY1” (also termed 6, NF-E1, UCRBP or CF1; refs. 45, 52, 56, 40, 69) is a cellular transcription factor that has been shown to bind to the RCS and inhibit LTR-driven HIV transcription. The protein and cDNA sequences for YY1 are set forth in FIG. 8 (SEQ ID NOS: 2 and 3).

As used herein, “LSF” (also termed LBF-1, CP-2, LBP-1a, b, c and d) is a lymphoid transcription factor that has been shown to repress LTR-driven transcription. The protein and cDNA sequences for LSF are set forth in FIG. 9 (SEQ ID NOS: 4 and 5).

As used herein, “HDAC1” refers to histone deacetylase 1, an enzyme that hydrolyzes n-acetyl groups on histones, nucleic proteins found in most eukaryotic cells. Histones are generally complexed to DNA in chromatin and chromosomes responsible for compacting DNA enough so that it will fit within a nucleus. Histones are generally of relatively low molecular weight and are basic, having a very high arginine/lysine content. They are highly conserved and may act as nonspecific repressors of gene transcription. The protein and mRNA sequences for HDAC1 are set forth in FIGS. 10A and 10B (SEQ ID NOS: 6 and 7).

### Proteins, Peptides, Derivative and Analogs:

The invention provides compositions comprising or, alternatively, consisting of or consisting essentially of, an isolated transcription repressor complex, comprised of YY1 and LSF, and HDAC1 proteins.

As used herein, a peptide is said to be “isolated” or “purified” when it is substantially free of homologous cellular material or chemical precursors or other chemicals. The peptides of the present invention can be purified to homogeneity or other degrees of purity. The level of purification will be based on the intended use.

The composition of the invention may contain a derivative (e.g., a fragment) or analog of YY1, LSF, and/or HDAC1.

As used herein, the terms “derivative” and “analog” refer proteins, the amino acid sequence of which consists of a portion the full length protein, which portion retains the activity of the full length protein. Activity or effectiveness of the proteins, derivatives and/or analogs of the invention for treatment or prevention of HIV infection can be determined by any of the methods by known in the art, disclosed herein and/or described in parent patent application, U.S. Ser. No. 09/355,010 and International Publication No. WO 98/33067, mentioned above and incorporated by reference in their entirety herein.

In the context of the instant invention, derivatives and/or analogs of the YY1, LSF and/or HDAC1 proteins retain activity sufficient to form the transcription repressor complex, bind to the RCS site of HIV LTR, and/or inhibit HIV transcription. In a preferred embodiment, the composition of the invention contains a YY1, LSF and/or HDAC1 derivative, the amino acid sequence of which consists of one or more functional domains of the YY1, LSF and/or HDAC1 proteins. In various specific embodiments, the portion of the YY1, LSF, and/or HDAC1 sequence is at least 10, 20, 30, 40, 50, 60, 80, 100, 200, 300, or 400 amino acids.

As used herein, a “functional domain” is the region of a protein (i.e., amino acid sequence) or an oligonucleotide (e.g., nucleic acid sequence) that is primarily responsible for the function of the protein (e.g., catalytic site of an enzyme, binding epitope of an antibody, etc.), the absence of which would result in loss of function.

The functional domain of YY1 has been analyzed and described by Bushmeyer et al. (Bushmeyer et al., 1995, J. Biol. Chem. 270:30213). The transcriptional repression domain was mapped in the region between amino acids 333 and 397 of the YY1 amino acid sequence (SEQ ID NO:2); therefore, it overlaps with the three last zinc finger domains. A more detailed mapping of the repression domain has been obtained in Yang Shi's laboratory. Interaction with other transcription factors has been shown to alter and regulate YY1 action (Seto et al., 1993, Nature 365:462; Lee et al., 1993, Proc. Natl. Acad. Sci. USA 90:6145; Lee et al., 1995, Nucleic Acid Research 23:925; Lee et al., 1995, Genes & Development 9:1188; Lewis et al., 1995, J. Virology 69:1628; Inouye and Seto, 1994, J. Biol. Chem. 269:6506). Several regions of YY1 have been shown to be involved in protein-protein interaction with transcription factors that regulate YY1 action: amino acids 260-331 are required for interaction with Sp1; amino acids 201-343 for interaction with c-Myc; amino acids 332-414 for interaction with E1A; and amino acids 224-330 and 332-414 are necessary for binding to ATF-2a (Bushmeyer et al., 1995, J. Biol. Chem. 270:30213; Zhou et al., 1995, J. Virology 69:4323). Further analysis of the LSF functional domain is provided in the Examples section below.

Preferred YY1 derivatives and analogs comprise the following sequences: amino acid numbers 50-414, 101-414, 150-414, 175-414, 200-414, 250-414, 260-414, 270-414, 280-414, 290-414, 300-414, 320-414, 340-414 and 360-414, and, most preferably, amino acid numbers 200-414, of the YY1 sequence as depicted in FIG. 8 (SEQ ID NO:3).

The LSF functional domains have been examined by Shirra et al. (Shirra et al., 1994, Molecular and Cellular Biology 14:5076). LSF binds the DNA as a tetramer. The region between amino acids 189 and 239 of the LSF amino acid sequence (SEQ ID NO:5) appears to be necessary for binding to the DNA. Further analysis of the LSF functional domain is provided in the Examples section below. For example, our studies show that truncations upstream of LSF amino acid 183 tend to disrupt the interaction between LSF and YY1. Likewise, interaction with YY1 appears to involve LSF amino acids 164-403.

In the context of the instant invention, preferred LSF derivatives and analogs containing the following amino acid sequences: amino acid numbers 150-250 and 200-300, and, most preferably, amino acid numbers 189-239 of the LSF sequence as depicted in FIG. 9 (SEQ ID NO:5). Preferred fragments are those less than 75, 100, 150, 200, 250, or 300 amino acids in length.

The HDAC1 functional domains relevant to its recruitment by YY1 are described herein, particularly in the Examples section, as well as in by W M Yang (71, 72).

In one embodiment, derivatives can be made by altering the amino acid sequence of the protein by substitutions, additions or deletions that provide for therapeutically effective molecules. Thus, the derivatives include peptides containing, as a primary amino acid sequence, all or part of the HDAC1, YY1 and/or LSF amino acid sequence including altered sequences in which functionally equivalent amino acid residues are substituted for residues within the sequence resulting in a peptide which is functionally active.

For example, one or more amino acid residues within the sequence can be substituted by another amino acid of a similar polarity which acts as a functional equivalent, resulting in a silent alteration. Conservative substitutions for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Any technique for mutagenesis known in the art can be used, including but not limited to, chemical mutagenesis, in vitro site-directed mutagenesis (Hutchinson et al., 1978, J. Biol. Chem. 253:6551), use of TAB7 linkers (Pharmacia), PCR with primers containing mutations, etc.

In certain embodiments, it is desirable to introduce nonclassical amino acids or chemical amino acid analogs as a substitution or addition into the HDAC1, YY1 and LSF proteins and/or derivatives. Non-classical amino acids include but are not limited to the D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

The invention also provides HDACT, YY1 and LSF, derivatives or analogs that are cyclized and/or branched using techniques known in the art

Also included within the scope of the invention are HDAC1, YY1 and LSF, or derivatives or analogs thereof, which are differentially modified during or after synthesis, e.g., by benzylation, glycosylation, acetylation, phosphorylation, amidation, pegylation, derivatization by known protecting/blocking groups, proteolytic cleavage, linkage to an antibody molecule or other cellular ligand, etc. In specific embodiments, the serine residues of YY1 and LSF, or derivatives or analogs thereof, are phosphorylated using techniques known in the art. In other specific embodiments, the YY1 and LSF, or derivatives or analogs thereof, are acetylated at the N-terminus and/or amidated at the C-terminus. Any of numerous chemical modifications may be carried out by known techniques, including but not limited to acetylation, formylation, oxidation, reduction; metabolic synthesis in the presence of tunicamycin; etc.

The derivative and/or analog may further comprise a chimeric, or fusion, protein comprising a component of the TRC (e.g., YY1, LSF, or HDACT) or a functional derivative or analog thereof joined at its amino- or carboxy-terminus via a peptide bond to an amino acid sequence of another HIV transcription factor, preferably one of the proteins of interest mentioned above. In a specific embodiment, the chimeric or fusion protein comprises an at least six amino acid portion, or an at least 10, 20, 30, 40, 50, 75, 100 or 200 amino acid portion, of one protein of interest joined via a peptide bond to an at least six amino acid portion, or an at least 10, 20, 30, 40, 50, 75, 100 or 200 amino acid portion, of another protein of interest preferably where said portions are active to treat or prevent HIV infection. Exemplary procedures for producing or expression such chimeric proteins are known in the art and/or described in parent patent application, U.S. Ser. No. 09/355,010 and International Publication No. WO 98/33067, mentioned above and incorporated by reference in their entirety herein.

HDAC1, YY1 and LSF derivatives and analogs can be made by chemical synthesis or by recombinant production from nucleic acid encoding HDAC1, YY1 and LSF peptide which nucleic acid has been mutated. Exemplary methods for chemically or recombinantly synthesizing proteins, derivatives and analogs are known in the art and/or fully described in parent patent application, U.S. Ser. No. 09/355,010 and International Publication No. WO 98/33067, mentioned above and incorporated by reference in their entirety herein.

### Nucleic Acids and Oligonucleotides:

The invention further provides nucleic acids comprising nucleotide sequences encoding YY1, or derivatives, fragments or analogs thereof; LSF, or derivatives fragments or analogs thereof; and HDAC1, or derivatives fragments or analogs thereof.

The nucleotide sequences encoding, and the corresponding amino acid sequences of, HDAC1, LSF and YY1 are known (Shi et al., 1991, Cell 67:377-388 and Kato et al., 1991, Science 251:1476, respectively) and are provided in FIGS. 8-10, respectively (SEQ ID NOS:2-7, respectively). Nucleic acids encoding HDAC1, LSF and YY1 can be obtained by any method known in the art, e.g., by PCR amplification using synthetic primers hybridizable to the 3′ and 5′ ends of the sequence and/or by cloning from a cDNA or genomic library using an oligonucleotide specific for the gene sequence.

Methodologies for identifying, isolating, and amplifying nucleotide sequences associated with HDAC1, LSF and YY1 are known in the art and/or fully described in parent patent application, U.S. Ser. No. 09/355,010 and International Publication No. WO 98/33067, mentioned above and incorporated by reference in their entirety herein.

The HDAC1, LSF or YY1 sequences provided by the instant invention include those nucleotide sequences encoding substantially the same amino acid sequences as found in native proteins, and those encoded amino acid sequences with functionally equivalent amino acids, as well as those encoding other derivatives or analogs.

Homologs (e.g., nucleic acids encoding HDAC1, LSF and YY1 of species other than human) or other related sequences (e.g., paralogs) can also be obtained by low, moderate or high stringency hybridization with all or a portion of the particular human sequence as a probe using methods well known in the art for nucleic acid hybridization and cloning. Exemplary hybridization procedures are known in the art and/or fully described in parent patent application, U.S. Ser. No. 09/355,010 and International Publication No. WO 98/33067, mentioned above and incorporated by reference in their entirety herein.

### Inhibitors, Antibodies, and Antisense:

The invention further provides for inhibitors of YY1, LSF and HDAC1 (or derivatives or analogs thereof), particularly those agents that (a) inhibit transcription repressor complex formation (i.e., the interaction between YY1, LSF and HDAC1); (b) inhibit the activity of one or more of the TRC components; (c) inhibit the binding of the TRC to the HIM LTR; and/or (d) prevent TRC-associated repression of HIV transcription. Such inhibitors may be identified by any method known in the art for assaying formation of the TRC; interaction among the TRC components; binding of the TRC to the HIV LTR or to its mediators; and/or HIV transcription, infection, or replication. Exemplary methods are known in the art, described herein and/or described in parent patent application, U.S. Ser. No. 09/355,010 and International Publication No. WO 98/33067, mentioned above and incorporated by reference in their entirety herein.

The compounds that may be screened in accordance with the invention include, but are not limited to, peptides, antibodies and fragments thereof, and other organic compounds (e.g., peptidomimetics) that inhibit formation of the TRC, activity of the TRC, or binding of the TRC to the LTR of HIV. These screens identify peptides, antibodies or fragments thereof, and other organic compounds that inhibit suppression of HIV transcription mediated by the TRC.

Such compounds may include, but are not limited to, peptides such as, for example, soluble peptides, including but not limited to, those found in random peptide libraries; (see, e.g., Lam at al., 1991, Nature 354:82-84; Houghten et al., 1991, Nature 354:84-86). Such compounds may also be found in combinatorial chemistry-derived molecular libraries made of D- and/or L-configuration amino acids; phosphopeptides (including, but not limited to, members of random or partially degenerate, directed phosphopeptide libraries; see, e.g., Songyang et al., 1993, Cell 72:767-778); antibodies (including, but not limited to, polyclonal, monoclonal, humanized, anti-idiotypic, chimeric or single chain antibodies, and FAb, F(abN)₂ and FAb expression library fragments, and epitope-binding fragments thereof); antisense RNA and small organic or inorganic molecules. In a specific embodiment, pyrrole-imidazole polyamides (e.g. as described in Gottesfeld et al., 1997, Nature 387:202-205) are provided to inhibit the activity of the transcription repressor complex on HIV gene expression.

By way of examples of non-peptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

In another embodiment of the invention, the TRC, protein components thereof, transcription mediators recruited thereby, or fragments, derivatives, or analogs these proteins, may be used as an immunogen to generate antibodies that recognize such an immunogen. Such antibodies include but are not limited to polyclonal, monoclonal, chimeric, single chain, Fab fragments, and an Fab expression library. In one embodiment, antibodies that specifically bind to YY1 or LSF and prevent TRC formation are provided. In another embodiment, antibodies that bind the TRC and prevent its binding to the HIV LTR are provided.

Various procedures known in the art may be used for the production of polyclonal antibodies and monoclonal antibodies to HDAC1, LSF, YY1, and/or the transcription repressor complex, or derivative or analog thereof. Exemplary procedures are described in parent patent application, U.S. Ser. No. 09/355,010 and International Publication No. WO 98/33067, mentioned above and incorporated by reference in their entirety herein.

Antibody fragments and other derivatives which contain the idiotype (binding domain) of the molecule can be generated by known techniques. For example, such fragments include but are not limited to: the F(abN)₂ fragment which can be produced by pepsin digestion of the antibody molecule; the FabN fragments which can be generated by reducing the disulfide bridges of the F(abN)₂ fragment; and the Fab fragments which can be generated by treating the antibody molecule with papain and a reducing agent.

The function of the TRC or the individual protein components thereof (e.g., LSF, YY1, and HDAC1) can be inhibited by use of antisense nucleic acids for HDAC1, LSF and/or YY1. The present invention provides the therapeutic or prophylactic use of nucleic acids of at least six nucleotides that are antisense to a gene or cDNA encoding HDAC1, LSF and/or YY1, or portions thereof.

An “antisense” nucleic acid as used herein refers to a nucleic acid capable of hybridizing to a sequence-specific (e.g., non-poly A) portion of the protein RNA (preferably mRNA) by virtue of some sequence complementarity. The antisense nucleic acid may be complementary to a coding and/or noncoding region of the protein mRNA. Such antisense nucleic acids have utility as therapeutics that inhibit transcription repressor complex formation or activity, or HDAC1, LSF, or YY1 function or activity, and can be used in the treatment or prevention of disorders as described supra.

The antisense nucleic acids of the invention can be oligonucleotides that are double-stranded or single-stranded, RNA or DNA or a modification or derivative thereof, which can be directly administered to a cell, or which can be produced intracellularly by transcription of exogenous, introduced sequences.

The antisense nucleic acids of the present invention are preferably at least six nucleotides and are more preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides.

The oligonucleotides can be DNA or RNA or chimeric mixtures or derivatives or modified versions thereof, single-stranded or double-stranded. In a preferred embodiment, the antisense oligonucleotide is a single-stranded DNA.

The oligonucleotide may be modified at any position on its structure with constituents generally known in the art. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652; PCT Publication No. WO 88/09810, published Dec. 15, 1988) or blood-brain barrier (see, e.g., PCT Publication No. WO 89/10134, published Apr. 25, 1988), hybridization-triggered cleavage agents (see, e.g., Krol et al., 1988, BioTechniques 6: 958-976) or intercalating agents (see, e.g., Zon, 1988, Pharm. Res. 5: 539-549). Exemplary modified moieties suitable for use with the antisense oligonucleotides of the present invention are known in the art and/or described in parent patent application, U.S. Ser. No. 09/355,010 and International Publication No. WO 98/33067, mentioned above and incorporated by reference in their entirety herein.

The oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art. Exemplary synthesis methods are described in parent patent application, U.S. Ser. No. 09/355,010 and International Publication No. WO 98/33067, mentioned above and incorporated by reference in their entirety herein.

The antisense oligonucleotides comprise catalytic RNAs, or ribozymes (see, e.g., PCT International Publication WO 90/11364, published Oct. 4, 1990; Sarver et alt, 1990, Science 247: 1222-1225). In another embodiment, the oligonucleotide is a 2N-0-methylribonucleotide (Inoue et al., 1987, Nucl. Acids Res. 15: 6131-6148), or a chimeric RNA-DNA analog (Inoue et al., 1987, FEBS Lett. 215: 327-330).

The antisense nucleic acids of the invention comprise a sequence complementary to at least a portion of an RNA transcript of the HDAC1, YY1, and/or LSF genes, preferably a human genes. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. One skilled in the art can ascertain a tolerable degree of mismatch by use of standard procedures to determine the melting point of the hybridized complex.

In another embodiment, the invention is directed to methods for inhibiting the expression of HDAC1, LSF and/or YY1 nucleic acid sequences in a cell comprising providing the cell with an effective amount of a composition comprising an antisense nucleic acid of LSF, YY1 and/or HDAC1, or derivatives thereof, of the invention. Cell types that express HDAC1, LSF or YY1 RNA can be identified by various methods known in the art. Such methods include, but are not limited to, hybridization with HDAC1, LSF and YY1-specific nucleic acids (e.g. by northern hybridization, dot blot hybridization, in situ hybridization), or by observing the ability of RNA from the cell type to be translated in vitro into HDAC1, LSF and YY1 by immunohistochemistry. In a preferred aspect, primary tissue from a patient can be assayed for HDAC1, LSF and/or YY1 expression prior to treatment, e.g., by immunocytochemistry or in situ hybridization.

### Enhancers, Activators and Regulators

The invention further provides for agents that affect or regulate the TRC-associated mechanism of repression. Techniques and procedures for identifying and screening for enhancers and regulators are analogous to those used to identify inhibitors, which are described and exemplified in the previous section.

As mentioned above, the invention provides for agents that inhibit, repress or down-regulate the TRC-associated mechanism of repression. Examples of such agents include: agents that repress or inhibit the formation of the TRC; agents that repress or inhibit the binding of YY1 (or a derivative or analog thereof) to HDAC1 (or a derivative or analog thereof; agents that repress or inhibit recruitment of HDAC1 (or a derivative or analog thereof) by YY1 (or a derivative or analog thereof) to the HIV LTR RCS site; agents that repress or inhibit the activity of HDAC1 (or a derivative or analog thereof); agents that repress or inhibit the expression of HDAC1 (or a derivative or analog thereof); and agents that down-regulate the expression of HDAC1 (or a derivative or analog thereof). These agents find particular therapeutic utility in blocking the repression of LTR-driven HIV transcription so as to treat or prevent of latent HIV infection.

In one embodiment, the regulator may comprise an inhibitor of the enzymatic activity of HDAC1. Exemplary HDAC1 inhibitors include trichostatin-A and trapoxin (Hassig C A et al., 1998, Proc Natl Acad Sci USA 95(7):3519-24).

In another embodiment, the regulator may comprise an agent that modulates expression and/or activity of HDAC1. For example, a dynamic balance of histone acetylation/deacetylation is maintained by histone acetyltransferases and histone deacetylases (Hu, E et al., 2000, J Biol Chem 275(20):15254-64).

In another embodiment, the regulator may comprise an agent that provides a post-translational modification (e.g., phosphorylation) to HDAC1, YY1 and/or LSF.

The invention further provides for agents that enhance, activate or up-regulate the TRC-associated mechanism of repression. Examples of such agents include: agents that enhance the formation of the TRC; agents that enhance the binding of YY1 (or a derivative or analog thereof) to HDAC1 (or a derivative or analog thereof); agents that enhance recruitment of HDAC1 (or a derivative or analog thereof by YY1 (or a derivative or analog thereof) to the HIV LTR RCS site; agents that enhance the activity of HDAC1 (or a derivative or analog thereof); agents that enhance the expression of HDAC1 (or a derivative or analog thereof); and agents that up-regulate the expression of HDAC1 (or a derivative or analog thereof). These agents find particular therapeutic utility in the repression of LTR-driven HIV transcription and the treatment or prevention of active HIV infection.

Enhancers and regulators, like inhibitors, may be identified by any method known in the art for assaying for formation of the TRC; interaction among the components of the TRC; binding of the TRC to the HIV LTR or to its mediators; and/or HIV transcription, infection, or replication. Exemplary methods are known in the art, described herein and/or described in parent patent application, U.S. Ser. No. 09/355,010 and International Publication No. WO 98/33067, mentioned above and incorporated by reference in their entirety herein. For example, a standard ELISA assay using an anti-HDAC1 antibody (such as that described in the Examples section below) as substrate may be used to measure the level of HDAC1 expressed in the presence (test) and absence (control) of a particular compound. A measured increase in expression in the presence of a compound is indicative of a compound's ability to enhance or up-regulate the expression of HDAC1. Likewise, co-precipitation assays (such as those described in the Examples section) using HDAC1 and YY1, for example, may be used to measure the binding affinity between the two molecules.

### Assays:

### Testing for Anti-HIV Activity

Anti-HIV activity of a therapeutic preparation can be measured by assaying it for the ability to inhibit HIV replication, transcription or expression of HIV RNA or protein. Any assay for HIV expression, replication or transcription, either in in vivo or in vitro, can be used to determine the level of transcription repression. For example, but not by way of limitation, EMSA for binding to the HIV LTR, the viral infection assays, CAT or other reporter gene transcription assays (with the CAT reporter gene or any other reporter gene known in the art operably linked to the HIV LTR), HIV infection assays, or assays for viral production from cells latently infected with HIV (for example, but not limited to, by the method described by Chun et al., 1977, Nature 387:183-188) can be used to screen for and test potential inhibitors of YY1-LSF complexes. Additional methods for assaying the efficacy of a particular preparation or therapy for HIV transcription repression are known in the art and/or described in parent patent application, U.S. Ser. No. 09/355,010 and International Publication No. WO 98/33067, mentioned above and incorporated by reference in their entirety herein.

The therapeutics of the invention are preferably tested in vitro, and then in vivo for the desired therapeutic or prophylactic activity, prior to use in humans. Any in vitro or in vivo assay known in the art to measure HIV infection, production, replication or transcription can be used to test the efficacy of a therapeutic of the invention. Exemplary in vitro or in vivo assays described herein as well as in parent patent application, U.S. Ser. No. 09/355,010 and International Publication No. WO 98/33067, mentioned above and incorporated by reference in their entirety herein.

In vivo animal models for testing the efficacy of the therapeutic preparation of the present invention are known in the art. Once the therapeutic preparation has been tested in vitro, and also preferably in a non-human animal model, the utility of the therapeutic preparation can be determined in human subjects. Exemplary human and animal assays are described in parent patent application, U.S. Ser. No. 09/355,010 and International Publication No. WO 98/33067, mentioned above and incorporated by reference in their entirety herein.

### Formation and Binding of Transcription Repressor Complex

TRC formation can be assayed by various methods, including but not limited to, protein affinity chromatography, affinity blotting, immunoprecipitation, cross-linking, and competitive inhibition assay methods (see generally, Phizicky et al., 1995, Microbiol. Rev. 59:94-123). Additionally, since the DNA encoding HDAC1, YY1 and LSF have been isolated and sequenced (see e.g., Shi et al., 1991, Cell 67:377-388 and Kato et al., 1991, Science 251:1476, respectively), this sequence may be routinely manipulated in known assays, to identify derivatives, fragments and analogs that bind counterpart members of the HIV-1 LTR binding, repressor complex. Such assays include, but are not limited to, in vitro cell aggregation and interaction trap assays (see generally, Phizicky et al., 1995, Microbiol. Rev. 59:94-123).

The affinity of derivatives and analogs for counterpart members of the repressor complex can routinely be determined by, for example, competitive inhibition experiments using HDAC1, YY1 and LSF, respectively. In specific embodiments, the derivatives or analogs of the invention display an affinity for the counterpart HIV-1 LTR binding complex member, which affinity approximates or is greater than the affinity of the protein from which it is derived.

The ability of complexes comprising YY1 (or derivatives and analogs thereof); LSF (or derivatives and analogs thereof); and HDAC1 (or derivatives and analogs thereof) to bind to the LTR of HIV-1 may routinely be determined using known assays, such as, for example, footprint and electrophoretic mobility shift assays (e.g., see Section 7, infra). These assays may routinely be applied to ascertain the affinity of the complex for DNA sequences of the LTR. In one preferred embodiment, the compositions of the invention containing YY1, LSF, and HDAC1 or derivatives and analogs thereof form complexes having the highest affinity for the DNA sequence of the HIV-1 LTR. In a further preferred embodiment, the compositions of the invention are form complexes that bind the DNA sequence corresponding to nucleotides −17 to +17 of the HIV-1 LTR as depicted in FIG. 7 (SEQ ID NO:1).

Transcriptional repression of HIV-1 by HDAC1, and derivatives and analogs thereof; LSF, and derivatives and analogs thereof; and YY1, and derivatives and analogs thereof, may routinely be examined using known techniques, such as, for may routinely be examined using known techniques, such as, for example, in vitro transcription experiments in which the HIV-1 LTR is operably linked to a reporter gene, such as, for example and not by way of limitation chloramphenicol acetyltransferase (CAT) (see e.g., Section 6 and 7, infra).

Assays for binding to the HIV LTR (e.g., electrophoretic mobility shift assay or EMSA) are also useful for testing the efficacy of therapeutic preparations of the invention. Specifically, the therapeutic preparations to be tested is incubated with radioactively labeled, double-stranded DNA containing the nucleotide sequence of −17 to +27 or −10 to +27 of the HIV LTR sequence and then analyzed by non-denaturing gel electrophoresis. A shift in the mobility of the labeled HIV LTR probe after incubation with the therapeutic preparation to be tested indicates that it binds to the HIV LTR.

### Screening Assays

Screening of compounds or compound libraries of putative inhibitors can be accomplished by any of a variety of commonly known methods described herein or in parent patent application, U.S. Ser. No. 09/355,010 and International Publication No. WO 98/33067, mentioned above and incorporated by reference in their entirety herein.

In a specific embodiment, screening can be carried out by contacting a single compound or multiple library members with an HDAC1, LSF or YY1 protein or derivative, or a repressor complex, or an HIV LTR nucleic acid immobilized on a solid phase and harvesting those library members that bind to the protein (or complex or nucleic acid or derivative).

In a specific embodiment, fragments and/or analogs of WI or LSF, especially peptidomimetics, are screened for activity as competitive or non-competitive inhibitors of YY1-LSF complex formation or binding of the complex to the HIV LTR, and thereby for the ability to inhibit YY1-LSF complex activity.

Numerous experimental methods may be used to select and detect proteins or non-protein molecules that interfere with the formation of the repressor complex or binding to the HIV LTR and thereby modulate HIV transcription including, but not limited to, protein affinity chromatography, affinity blotting, immunoprecipitation, cross-linking, and library based methods such as protein probing, phage display and the two-hybrid system. See generally, Phizicky et al., 1995, Microbiol. Rev. 59:94-123. For example, the two-hybrid system may be used to detect inhibitors of the interaction between LSF and YY1 by constructing the appropriate hybrids and testing for reporter gene activity in the presence of candidate inhibitors.

Any assay for HIV infection, replication or transcription, either in in vivo or 117 vitro, can be used to screen for inhibitors of the YY1-LSF complex activity.

In the production of antibodies, screening for the desired antibody can be accomplished by techniques known in the art, e.g. ELISA (enzyme-linked immunosorbent assay). For example, to select antibodies that recognize a specific domain of LSF, YY1 or a YY1-LSF complex, one may assay generated hybridomas for a product that binds to a fragment containing such domain. For selection of an antibody specific to human LSF, YY1 or YY1-LSF complex, one can select on the basis of positive binding to a human protein or complex and a lack of binding to the protein or complex of another species, e.g. mouse, rat, primate, etc.

### Therapeutic Utilities:

The invention provides for repression of HIV transcription to treat diseases and disorders associated with HIV infection by administration of a therapeutic compound (termed herein “therapeutic”). Such “therapeutics” include, but are not limited to: compositions containing YY1, LSF, and HDAC1 and therapeutically and prophylactically effective derivatives (including fragments) and/or analogs thereof, i.e., those derivatives and/or analogs which prevent or treat HIV infection (e.g., as demonstrated in in vitro and in vivo assays described infra), as well as nucleic acids encoding YY1, LSF, and HDAC1, and/or therapeutically and prophylactically effective derivatives and analogs thereof (e.g., for use in gene therapy); modulators (e.g., antagonists, inhibitors and agonists) of the activity of YY1, of LSF, of HDAC1 or of the transcription repressor complexes containing HDAC1, YY1 and LSF, e.g., but not limited to, antibodies against HDAC1, YY1, LSF or the TRC containing YY1 and LSF; HDAC1, YY1 and/or LSF antisense nucleic acids, organic and inorganic small molecules such as peptides, peptidominetics, polyamides (e.g., those described by Gottesfeld et al., 1997, Nature 387: 202-205), etc. Examples of therapeutics are those proteins, derivatives and analogs of YY1, LSF and HDAC1, as well and inhibitors of the TRC activity, described above. Additional examples are set forth in parent patent application, U.S. Ser. No. 09/355,010 and International Publication No. WO 98/33067, mentioned above and incorporated by reference in their entirety herein.

A preferred embodiment of the invention relates to methods of using a therapeutic for treatment of HIV infection, preferably HIV-1 infection, in a human subject. The therapeutic of the invention can be used to prevent progression of HIV-1 infection to ARC or to AIDS in a human patient, or to treat a human patient with ARC or AIDS.

In a specific embodiment, the therapeutic method of the invention is carried out as monotherapy, i.e., as the only agent provided for treatment of HIV. In another embodiment, the therapeutic is administered in combination with one or more anti-viral compounds, for example, protease inhibitors (e.g., saquinavir, indinavir, ritonavir, nelfinavir) and/or reverse transcriptase inhibitors (e.g., azidothymidine (AZT), lamivudine (3TC), dideoxyinosine (ddI), dideoxycytidine (ddC), nevirapine, and efavirenz). The therapeutic may also be administered in conjunction with chemotherapy (e.g., treatment with adriamycin, bleomycin, vincristine, vinblastine, doxorubicin and/or Taxol) or other therapies known in the art.

In another embodiment, HIV infection is treated by administration of a therapeutic of the invention in combination with one or more chemokines. In particular, the therapeutic is administered with one or more C—C type chemokines, especially one or more from the group RANTES, MIP-1α, MIP-1β and MDC, or the C—X—C type chemokine, SDF-1.

In another embodiment, HIV infection is treated by administration of a combination of one or more transcription factor therapeutics and one or more HIV protein therapeutics of the invention.

In another embodiment, HIV infection is treated by administration of a therapeutic of the invention to antagonize transcriptional repression of the HIV1 LTR. Examples of such therapeutics are discussed parent patent application, U.S. Ser. No. 09/355,010 and International Publication No. WO 98/33067, mentioned above and incorporated by reference in their entirety herein.

One aspect of the invention relates to assaying preparations of YY1 and LSF, and/or derivatives and/or analogs thereof, for efficacy in treatment or prevention of HIV infection. The therapeutic effectiveness of these preparations can be tested by the in vitro or in vivo assays described in or by any method known in the art for assaying HIV infection, transcription or replication. It is preferable to test the preparation in an in vitro assay, e.g., for HIV infection, replication, transcription from the HIV LTR or binding to the HIV LTR by an EMSA, or in vivo in an animal model, such as HIV transgenic mice or SIV infected monkeys, before assaying the preparation in humans.

In a specific embodiment, a preparation comprising YY1, LSF and HDAC1 is used.

The HDAC1, YY1 and LSF-related polypeptides are preferably prepared by any chemical or enzymatic synthesis method known in the art, as described above.

The invention provides methods of treatment and prevention by administration to a subject in need of such treatment of a therapeutically or prophylactically effective amount of a therapeutic of the invention. The subject is preferably an animal, including, but not limited to, animals such as monkeys, cows, pigs, horses, chickens, cats, dogs, etc., and is preferably a mammal, and most preferably human.

Various delivery systems are known and can be used to administer a therapeutic of the invention, e.g., encapsulation in liposomes, microparticles, microcapsules, recombinant cells capable of expressing the therapeutic, receptor-mediated endocytosis (see, e.g., Wu and Wu, 1987, J. Biol. Chem. 262:4429-4432), construction of a therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

Such delivery systems, administration routes, and components thereof are known in the art and described in parent patent application, U.S. Ser. No. 09/355,010 and International Publication No. WO 98/33067, mentioned above and incorporated by reference in their entirety herein.

Where the therapeutic is a nucleic acid encoding a protein therapeutic, the nucleic acid can be administered by gene therapy methods as herein, known in the art and/or described in parent patent application, U.S. Ser. No. 09/355,010 and International Publication No. WO 98/33067, mentioned above and incorporated by reference in their entirety herein.

### Pharmaceutical Compositions:

The present invention also provides pharmaceutical compositions. Such compositions comprise a therapeutically effective amount of a therapeutic, and a pharmaceutically acceptable carrier.

The term “Pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and can be determined by standard clinical techniques. In addition, in vivo and/or in vitro assays may optionally be employed to help predict optimal dosage ranges. The precise dose to be employed in the formulation will also depend on the route of administration, and the seriousness of the disease or disorder, and should be decided according to the judgment of the practitioner and each patients circumstances. Routes of administration of a therapeutic include, but are not limited to, intramuscularly, subcutaneously or intravenously. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, the pharmaceutical compositions of the invention may contain an effective amount of an HDAC1, LSF and/or YY1 antisense nucleic acid in a pharmaceutically acceptable carrier can be administered to a patient having a disease or disorder which is of a type that expresses the YY1-LSF-HDAC1 complexes or HDAC1, LSF or YY1 RNA or protein.

The amount of HDAC1, LSF and/or YY1 antisense nucleic acid that will be effective in the treatment of a particular disorder or condition will depend on the nature of the disorder or condition, and can be determined by standard clinical techniques. Where possible, it is desirable to determine the antisense cytotoxicity in vitro, and then in useful animal model systems prior to testing and use in humans.

In a specific embodiment, antisense pharmaceutical compositions can be administered via liposomes, microparticles, or microcapsules. In various embodiments of the invention, it may be useful to use such compositions to achieve sustained release of the antisense nucleic acids. In a specific embodiment, it may be desirable to utilize liposomes targeted via antibodies to specific identifiable cell types (Leonetti et al., 1990, Proc. Natl. Acad. Sci. U.S.A. 87: 2448-2451; Renneisen et al., 1990, J. Biol. Chem. 265: 16337-16342), e.g. to HIV infected T cells.

### EXAMPLES

### Materials And Methods

Nuclear extracts. Large-scale preparation of nuclear extract from CEM cells for chromatographic purification of the TRC were prepared as described (12), with the following minor modifications: buffer A and C were supplemented with 1 mM NaF, 1 mM Na₂VO₄, 10 μg of leupeptin per ml, 10 μg of aprotinin per ml, 1 μg of pepstatin A per ml. Chymostatin (1 μg/ml) was also added to buffer A and 50 mM beta-glycerophosphate to buffer C.

Ion-exchange chromatography. Activated P11 phosphocellulose (Whatman, Clifton, N.J.) was equilibrated with 50 mM NaCl, 50 mM HEPES (pH 7.9), 10% glycerol, 0.2 mM EDTA, 0.5 mM phenylmethylsulfonyl fluoride (PMSF) and 0.5 mM dithiothreitol (DTT). CEM cell nuclear extract was loaded at 0.4 ml/min, washed, and eluted in a linear gradient of 50 mM to 1 M NaCl. Fractions shown by Western blot with anti-YY1 C-20 antibody (Santa Cruz Biotechnology, Santa Cruz, Calif.) to contain YY1, and shown by EMSA to contain RCS binding activity were pooled and dialyzed against 20 mM Tris-HCl (pH 7.9), 10% glycerol, 0.2 mM EDTA, 0.5 mM PMSF, 0.5 mM OTT, and 50 mM NaCl before DEAF-cellulose chromatography. A DEAF-cellulose DE52 column (Whatman, Clifton, N.J.) was loaded with pooled fractions at 0.2 ml/min. The column was washed and eluted, and fractions analyzed as above. Fractions positive both in Western blot and gel shift were subjected to further purification by DNA-affinity chromatography.

DNA-affinity chromatography. A double stranded oligonucleotide spanning the region −10 to +27 of the HIV-1 LTR was ligated and coupled to CNBr-activated Sepharose CL-4B (Pharmacia, Piscataway, N.J.) as previously described (27). Active fractions from DEAE-cellulose chromatography were equilibrated in buffer Z (25 mM HEPES [pH 7.6], 0.1 M NaCl, 20% glycerol, 12.5 mM MgCl₂, 1 mM DTT, 0.5 mM PMSF, 0.1% Nonidet P-40). Affinity resin was washed extensively with buffer Z without glycerol and Nonidet P-40. Fractions were incubated for 10 minutes at 4° C. with 10 μg of (dI-dC) per ml, loaded by gravity, washed, and eluted with a step gradient of 0.1 to 1 M NaCl. Western blot analysis for detection of histone deacetylase was performed using a rabbit polyclonal antibody raised against a peptide corresponding to the 319-334 C-terminal amino acids of the molecule (59), and against LSF using rabbit polyclonal anti-CP2 antiserum (LSF, LBP-1c; gift of M. Sheffery). Histone deacetylase assays were performed as previously described (31).

Cell lines transfections and assays. Transfections of HeLa cells were performed as previously described (49). HeLa-CD4-LTR (9) cells were grown in DMEM supplemented with 10% FCS and transfected with 20 μg plasmid DNA (prepared using EndoFree plasmid kit, Qiagen, Valencia, Calif.) by calcium phosphate coprecipitation as per manufacturer's instructions (ProFection system, Promega, Madison, Wis.). After 30 minutes at room temperature, the solution was added to the cells (2.5-4×10⁵ cells/plate). Twelve hours after transfection, the cells were washed with PBS and fed fresh medium. Forty-eight hours later the cells were harvested, cellular extracts prepared, and CAT assays performed as previously described (49). To control for the effect of transcription factor overexpression on general cellular promoters a reporter construct driven by the beta-actin promoter, pH□-actin-luciferase (66), was used in cotransfection and CAT expression normalized for luciferase activity. Other plasmids used have been previously described (2, 49). Luciferase assays were performed at 48 hours as suggested by the manufacturer (Luciferase assay system, Promega, Madison, Wis.) but cells resuspended in 200 μl of lysis buffer and one freeze/thaw step performed. Up to 30 μl of cellular extract (normalized for protein concentration) in a final volume of 130 μl was used for luciferase reactions.

For virus production experiments 2×10⁴ HeLa cells were transfected with 10 μl of Superfect (Qiagen, Valencia, Calif.) and 2.5-3.0 μg of DNA in a volume of 0.6 ml for 3 hours, washed with PBS, and then grown in 2 ml. Aliquots of culture medium were sampled for detection of HIV-1 p24^{gag} protein by antigen capture enzyme-linked immunosorbent assay according to the manufacturer's instructions (Coulter Corporation, Hialeah, Fla.).

Immunoprecipitation and electrophoretic mobility shift assays (EMSA): Immunoprecipitation was performed using nuclear extracts prepared from a Jurkat T cell line (49). 20 μl samples of extract were mixed with antibody (rabbit IgG, −YY1 {C20; Upstate Biotechnology, Lake Placid, N.Y.} or α-LSF {a gift of M. Sheffery and S. Swendenmann}) at 4° C. for 1 hour. 5 μl α-rabbit IgG agarose conjugated antibody (Sigma, St. Louis, Mo.) was added and incubation was continued for 1 hour. The complex was precipitated by centrifugation at 3000 rpm, 4° C. for 5 minutes. The pellet was washed 3 times with PBS, resuspended in 30 μl SDS-PAGE sample buffer before separation by SDS-PAGE and Western analysis with α-YY1 or α-LSF antibodies. Bands were visualized using HRP conjugated α-rabbit IgG (Sigma).

Histidine-tagged YY1 (his-YY1) and histidine-tagged LSF (his-LSF) were expressed and harvested in E. coli as previously described (53, 64). The RCS oligonucleotide (−10 to +27 of HIV-1 LTR, ref. 49) was end labeled and 1×10⁵ cpm was incubated with varying amounts of his-LSF for 20 min at 25° C. GST-YY1 or his-YY1 was added, total protein content normalized by the addition of BSA, and the reaction continued for 30 minutes. EMSA was then performed as previously described (49). Supershifts were performed by addition of either concentrated □-YY1 (C20-X; Santa Cruz Biotech, Santa Cruz, Calif.) or α-LSF (64) to the reaction mixture.

In vitro protein interaction mapping: Glutathione-S-transferase (GST)-YY1/GFI-1 chimeras (17) and G3ST-LSF deletion (54, 55) constructs were expressed and harvested in E. coli as previously described. Proteins were visualized by Coomassie brilliant blue staining and protein content was normalized by densitometric analysis. LSF was transcribed and translated in vitro by T7 RNA polymerase in rabbit reticulocyte (Promega, Madison, Wis.) in the presence of ³⁵S-methionine as per manufacturer's instruction (54). Following capture of GST-YY1/GFI-1 chimera proteins on glutathione-agarose beads, equal volumes of beads were incubated with ³⁵S-methionine labeled LSF in incubation buffer (17) for 1 hour at 4° C. The beads were washed in incubation buffer/100 mM KCl and resuspended in 20 μl 2×SDS loading buffer. Retained LSF was separated in a 10% SDS gel, dried, and visualized by autoradiography. Similarly, YY1 was transcribed and translated in vitro, incubated with captured GST-LSF constructs, and retained YY1 was by autoradiography following electrophoresis.

### Example 1

### YY1 and LSF Interact In Vivo

Complex formation at the RCS site can be ablated by either α-YY1 or α-LSF, suggesting that both YY1 and LSF are necessary to form this regulatory complex (49). In this example, we describe evidence that these factors interacted directly in the absence of a DNA binding sequence.

Jurkat CD4⁺ T cell nuclear extracts were incubated with α-YY1, α-LSF, or a nonspecific rabbit polyclonal antiserum. Antibody-protein complexes were precipitated by addition of α-IgG-agarose beads and centrifugation. Precipitates were then assayed for the presence of LSF by Western blot analysis. Immunoprecipitation was specific, as only trace amounts of LSF were recovered by the nonspecific antiserum. α-YY1 precipitated approximately 75% of the LSF activity that could be recovered by α-LSF (FIG. 1a). This indicates that LSF interacts with YY1 in vivo in the absence of the HIV RCS binding site and suggests that direct protein-protein interaction between YY1 and LSF is necessary for complex formation at the HIV LTR.

### Example 2

### LSF and YY1 Associate without Cofactors In Vitro

Romerio et al (49) showed that both LSF and YY1 form a complex at the RCS site of the HIV LTR. It was not known whether LSF and YY1 were sufficient to form this complex. We reconstituted this complex in vitro, in the absence of other factors to address this question.

Histidine-tagged recombinant LSF and YY1 proteins were expressed, harvested from E. coli, and allowed to interact with an oligonucleotide encoding the HIV LTR RCS (SEQ ID NO. 1). Up to 100 ng of His-YY1 alone did not form a detectable complex with the RCS (FIG. 1b). Whereas 10 ng of LSF formed a diffuse faintly visible EMSA band (FIGS. 1b and 1c), the addition of increasing amounts (20 ng to 100 ng) of YY1 resulted in increasing levels of complex formation in the presence of 10 ng of LSF (FIG. 1b). As little as 20 ng of His-LSF induced a very prominent protein-DNA complex (FIG. 1c), similar to previous studies (18, 26). Under these conditions, no additional effect of YY1 was discernable when added to 20 ng of LSF. However YY1 specifically enhanced TRC formation, as all reactions were normalized for total protein content by the addition of BSA.

These protein-DNA complexes were supershifted by either α-YY1 or (X-LSF antibodies (FIG. 1c), confirming that these complexes contained both LSF and YY1. α-LSF completely shifted the TRC, whereas under these conditions only a fraction of complexes were supershifted by the addition of excess α-YY1. The quantity of α-YY1 (5 μl) completely supershifted YY1 complexes formed on a canonical AAV P5 binding site (data not shown). The effect of the antibody was specific, as α-YY1 had no effect on the mobility of the complex in the absence of YY1 protein (data not shown). Although other factors may be present in the TRC in vivo, YY1 and LSF are sufficient to form a complex at the RCS.

The addition of YY1 to LSF bound to the RCS was not associated with a further change in the mobility of the DNA-protein complex.

### Example 3

### Interaction Domains of YY1 and LSF

The interaction of LSF with YY1 was mapped using constructs that contained a series of nested deletions within the coding region of LSF (FIG. 2a).

Neither the carboxyl nor amino terminus of LSF was required for interaction with YY1. However, the central region of the protein was required for interaction with YY1, as amino terminal deletions beyond amino acid 164, and carboxy terminal deletions prior to amino acid 403 resulted in marked diminution of the ability of LSF to bind YY1 (FIG. 2b). Binding was lost altogether when c-terminal sequences between amino acid residues 308 and 368 were removed. Further amino acid substitutions within this region, which impair multimerization (55), markedly decreased the ability of the mutant LSF to bind full length YY1. A likely possibility is that LSF recognizes YY1 only in its multimeric conformation.

YY1 DNA-binding activity and many YY1-protein interactions map to the carboxyl-terminal zinc fingers of the molecule. Therefore, YY1 interaction domains were mapped using chimeric YY1 recombinants (FIG. 2c). These chimeras expressed GST fused to the N-terminal domain of the protein and had varying numbers of YY1 zinc finger domains replaced by the structurally homologous GFI-1 zinc fingers (17). No deleterious effect on function or stability of YY1 was observed (17). FIG. 2c shows that the interaction with LSF the third and fourth zinc fingers of YY1 are not required to retain LSF. LSF bound in vitro to constructs that contained YY1 zinc fingers 1, 2, or both. In these assays chimera 2, which expresses zinc fingers 1 and 2 bound LSF more avidly than intact YY1. Chimera 1 bound LSF nearly as well as YY1, while chimera 7 could bind LSF in vitro, but less avidly. Thus, either zinc finger allowed binding to LSF, but binding was optimal when both fingers were present. Chimera 5, containing only the last two zinc fingers of YY1, did not bind LSF. The GST-Y/GFI-1 construct containing the entire YY1 amino-terminal domain fused to the zinc finger domain of the GFI-1 protein retained minimal amounts of LSF. Finally, a chimera expressing only the YY1 zinc finger domains, and lacking the entire amino terminal YY1 region, bound LSF at least as well as intact YY1. Therefore, YY1 requires only zinc fingers 1 and 2 to recognize LSF.

### Example 4

### LSF Competent to Bind DNA is Required for Repression of HIV LTR Expression

Shirra and Hansen (54) and Shirra et al (55) demonstrated that LSF binds a canonical SV40 late promoter site via the formation of homotetramers. Further, binding can be blocked by the expression of a dominant negative mutant defective in DNA binding but with remaining ability to multimerize (LSF 234QL/236KE or dnLSF). The following experiments were performed to test the effect of dnLSF using the HeLa-CD4-LTR cell line (9).

The LTR reporter carried by HeLa-CD4-LTR cell line exists within the native chromatin structure of the genome. Transfection of YY1 inhibited Tat-activated CAT activity in these cells (FIG. 3) in agreement with previous studies using plasmid-based reporters (49). In the setting of a chromosomally integrated reporter gene the provision of LSF augmented repression mediated by YY1, confirming the effect of YY1 and LSF on an integrated HIV-1 promoter. Significantly, dnLSF abolished the ability of YY1 to repress CAT expression, confirming that LSF capable of binding DNA is required to allow YY1 to repress HIV-1 LTR expression (FIG. 3). As in previous studies (49), these effects were specific to the HIV LTR; results are normalized to the expression of a cotransfected beta-actin-luciferase reporter gene, whose expression was not significantly affected by YY1, LSF, or dnLSF (not shown).

### Example 5

### Copurification of HDAC1 with the YY1-LSF Complex

YY1 has been demonstrated to act via the recruitment of histone deacetylases (71, 72). As a nucleosome is present near the RCS when the HIV-1 LTR is integrated (46, 51, 61), we tested to see if histone deacetylase was present in the RCS DNA affinity chromatography fractions.

The YY1-LSF complex was purified by phosphocellulose P11, DEAE-cellulose column, and DNA affinity chromatography, as previously described (49). YY1, LSF and the RCS-binding activity copurified in the 0.3 and 0.4 M NaCl fractions of the final step of purification (FIG. 4a). RCS-binding activity was enriched about 10,000-fold by this procedure. As shown in FIG. 4a, a rabbit polyclonal antibody raised against amino acids 319-334 of HDAC1 was able to detect a protein with apparent molecular mass of 66 kDa in the 0.3 and 0.4 M NaCl pooled fractions. HDAC1, a 55 kDa protein, migrates at this apparent molecular mass in our gel system (58b).

To rule out the possibility that these fractions contained a protein immunologically similar but enzymatically unrelated to histone deacetylase, we assayed the histone deacetylase activity of the DNA affinity chromatography fractions. As expected, the 0.3 and 0.4 NaCl fractions showed strong HDAC1 activity, as measured by release of ³H-acetic acid (FIG. 4b). These results indicate that active HDAC1 copurifies with the YY1-LSF complex, and suggests that the YY1-LSF complex represses HIV-1 transcription via the recruitment of HDAC1.

### Example 6

### Repression of the HIV-1 LTR by YY1 Requires Interaction with a Histone Deacetylase

A recent report has demonstrated that the Gly/Ala-rich domain of YY1 mediates the interaction with the histone deacetylase, and is required for repression of the adeno-associated virus (AAV) P5 promoter by YY1 (71). To test whether the mechanism of repression of the HIV-1 LTR by YY1 is mediated by a histone deacetylase, we performed a series of transient transfection experiments using a mutant YY1 deleted of the Gly/Ala-rich domain (YY1Δ154-199; ref. 2) required for interaction with HDAC1.

Initial experiments were performed as previously (41, 49), demonstrating that cotransfection of YY1 inhibited Tat-activated, LTR-driven CAT expression. However, YY1Δ154-199 was unable to inhibit CAT activity, indicating the absolute requirement of the Gly/Ala-rich domain of YY1 for efficient repression of the HIV-1 LTR (data not shown). Indeed, YY1Δ154-199 activated LTR expression, suggesting ongoing competition between constitutive cellular YY1 and HIV LTR activating factors.

Chromatin remodeling effects on gene activity have often been imputed in studies using transfected, plasmid-encoded reporter genes that may not reflect the activity of genes contained in native chromatin. However the LTR reporter carried by the HeLa-CD4-LTR cell line (9) exists within the native chromatin structure of the genome. Significantly, YY1Δ154-199 failed to repress CAT expression, confirming that the Gly/Ala-rich histone deacetylase interaction domain is required for repression of the HIV-1 LTR by YY1 (FIG. 3). Indeed, YY1□154-199 activated CAT expression, but when normalized for modest activation of a cotransfected beta-actin-luciferase reporter, this effect was not significant. Repression was also blocked by addition of the specific HDAC1 inhibitor trichostatin-A to the culture medium (data not shown).

This is the first demonstration of both cooperative repression by YY1 and LSF, and the lack of repression by YY1Δ154-199 in the context of a chromosomally integrated HIV-1 promoter. It is also the first demonstration in this context that YY1 requires its HDAC1 interaction domain to mediate repression.

### Example 7

### Repression of the HIV-1 Virion Production by YY1 Requires Interaction with a Histone Deacetylase

Support for the role of HDAC1 in repression of HIV-1 virion production was demonstrated by cotransfection of HeLa cells with the infectious molecular clones pNL4-3 (1) or pYU-2 (37) and empty CMV vector, CMV-YY1, or CMV-YY1Δ154-199. As these cells support HIV replication but cannot be infected, a measurement of the effect of YY1 on a single round of viral replication can be made.

The influence of YY1 on viral production was assayed by testing of culture supernatant for the presence of the viral protein p24^{gag}. Cotransfection with a vector expressing YY1 produced dose-dependent inhibition of either CXCR4 (pNL4-3) or CCR5 (pYU-2) tropic virus, whereas cotransfection of YY1Δ154-199 failed to inhibit HIV production (FIG. 5). Again, YY1Δ154-199 activated HIV expression above normal levels. Similar results were seen in the CD4⁺ T cell line CEM when transfected with pNL4-3 or pYU-2 and empty CMV vector, CMV-YY1, or CMV-YY1Δ154-199 (data not shown). These findings suggest the possibility of ongoing competition between constitutive cellular YY1 and HIV LTR activating factors, although secondary activating effects of CMV-YY1Δ154-199 cannot be excluded.

While the invention has been described in conjunction with examples thereof, it is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

All references cited herein are incorporated by reference in their entirety.

### REFERENCES

1. Adachi, A., H. E. Gendelman, S. Koenig, T. Folks, R. Willey, A. Rabson, and M. A. Martin. 1986. Production of acquired immunodeficiency syndrome-associated retrovirus in human and non-human cells transfected with an infectious molecular clone. J. Virology. 59:284-291.
2. Austen, M., B. Lüscher, and J. M. Lüscher-Firzlaff. 1997. Characterization of the transcriptional regulator YY1. J. Biol. Chem. 272:1709-1717. Biochem. Sci. 19: 38-42.
3. Brehm, A., E. A. Miska, D. J. McCance, J. L. Reid, A. J. Bannister, & T. Kouzarides. 1998. Retinoblastoma protein recruits histone deacetylase to repress transcription. Nature (London) 391: 597-601.
4. Bushmeyer, S., K. Park, & M. L. Atchison. 1995. Characterization of functional domains within the multifunctional transcription factor, YY1. J. Biol. Chem. 270: 30213-30220.
5. Chiang, C.-M. and R. G. Roeder. 1995. Cloning of an intrinsic human TFIID subunit that interacts with multiple transcriptional activators. Science. 267:531-535.
6. Chun, T.-W., D. Finzi, J. Margolick, K. Chadwick, D. Schwartz, and R. F. Siliciano. 1995. In vivo fate of HIV-1-infected T cells: quantitative analysis of the transition to stable latency. Nature Med 12:1284-1290.
7. Chun T.-W., L. Carruth, D. Finzi, X. Shen, J. A. DiGiuseppe, H. Taylor, M. Hermankova, K. Chadwick, J. Margolick, T. C. Quinn, Y. H. Kuo, R. Brookmeyer, M. A. Zeiger, P. Barditch-Crovo, and R. F. Siliciano. 1997. Quantification of latent tissue reservoirs and total body viral load in HIV-1 infection. Nature 387:183-188.
8. Chun T.-W., L. Stuyver, S. B. Mizell, L. A. Ehler, J. A. Mican, M. Baseler, A. L. Lloyd, M. A. Nowak, A. S. Fauci. (1997). Presence of an inducible HIV-1 latent reservoir during highly active antiretroviral therapy. Proc Natl Acad Sci USA 94:13193-7.
9. Ciminsale, V., B. K. Felber, M. Campbell, and G. N. Pavlakis. 1990. A bioassay for HIV-1 based on Env-CD4 interaction. AIDS Res. Hum. Retroviruses. 6: 1281-1287.
10. Cowell, I. G. 1994. Repression versus activation in the control of gene transcription. Trends Biochem. Sci. 19: 38-42.
11. Davie, J. R. 1998. Covalent modifications of histones: expression from chromatin templates. Curr. Opin. Genet. Dev. 8: 173-178.
12. Dignam, J. D. 1990. Preparation of extracts from higher eukaryotes. Methods Enzymol. 182: 194-203.
13. El-Kharroubi, A., G. Piras, R. Zensen, and M. A. Martin. 1998. Transcriptional activation of the integrated chromatin associated human immunodeficiency virus type 1 promoter. Mol Cell Biol. 18:2535-44.
14. Felsenfeld, G. 1996. Chromatin unfolds. Cell. 86:13-19.
15. Finzi, D., M. Hermankova, T. Pierson, L. M. Carruth, C. Buck, R. E. Chaisson, T. C. Quinn, K. Chadwick, J. Margolick, R. Brookmeyer, J. Gallant, M. Markowitz, D. D. Ho, D. D. Richman, R. F. Siliciano. (1997). Identification of a reservoir for HIV-1 in patients on highly active antiretroviral therapy. Science 278:1295-300.
16. Flanagan, J. R. 1995. Autologous stimulation of YY1 transcription factor expression: role of an insulin-like growth factor. Cell Growth and Diff. 6: 185-190.
17. Galvin, K. M., and Y. Shi. 1997. Multiple mechanisms of transcriptional repression by YY1. Mol. Cell. Biol. 17: 3723-3732.
18. Garcia, J. A., F. K. Wu, R. Mitsuyasu, and R. B. Gaynor, 1987. Interactions of cellular proteins involved in the transcriptional regulation of human immunodeficiency virus. EMBO. 6: 3761-3770.
19. Geisberg, J. V., W. S. Lee, A. J., Berk, and R. P. Ricciardi. 1994. The zinc finger region of the adenovirus E1A transactivating domain complexes with the TATA box binding protein. Proc. Natl. Acad. Sci. USA. 91: 2488-2492.
20. Grunstein, M. 1997. Histone acetylation in chromatin structure and transcription. Nature (London) 389: 349-352.
21. Hassig, C. A., T. C. Fleiseher, A. N. Billin, S. L. Schreiber, & D). E. Ayer. 1997. Histone deacetylase activity is required for full transcriptional repression by mSin3A. Cell. 89: 341-347.
22. Huang, H.-C., R. Sundseth, and U. Hansen. 1990. Transcription factor LSF binds two variant bipartite sites within the SV40 late promoter. Genes Dev. 4:287-298.
23. Inouye, C. J., and EC. Seto. 1994. Relief of YY1-induced transcriptional repression by protein-protein interaction with the nucleolar phosphoprotein B23. J. Biol. Chem. 270:15187-93.
24. Johnson, A. D. 1995. The price of repression. Cell. 81: 655-658.
25. Johnson, C. A. and B. M. Turner. 1999. Histone Deacetylases: complex transducers of nuclear signals. Semin Cell Dev Biol. 10(2), 179-88.
26. Jones, K. A., P. A. Luciw, and N. Duchange. 1988. Structural arrangements of transcription control elements within the 5′-untranslated leader regions of the HIV-1 and HIV-2 promoters. Genes Dev. 2:1101-1114.
27. Kadonaga, J. T. 1991. Purification of sequence-specific binding proteins by DNA affinity chromatography. Methods Enzymol. 208: 10-23.
28. Kadosh, D., & K. Struhl. 1997. Repression by Ume6 involves recruitment of a complex containing Sin3 corepressor and Rpd3 histone deacetylase to target promoters. Cell. 89: 365-371.
29. Kato, H., M. Horikoshi, and R. G. Roeder. 1991. Repression of HIV-1 transcription by a cellular protein. Science 251:1476-1479.
30. Kurokawa, M., K. Mitani, K. Irie, T. Matsuyama, T. Takahashi, S. Chiba, Y. Yazaki, K. Matsumoto, and H. Hirai. 1998. The oncoprotein Evi-1 represses TGF-beta signaling by inhibiting Smad3. Nature. 394: 92-96.
31. Laherty, C. D., W.-M. Yang, J.-M. Davie, J. R. Sun, E. Seto, and R. N. Eisenmann. 1997. Histone deacetylase associated with the mSin3 corepressor mediate Mad transcriptional repression. Cell. 89:349-356.
32. Lee, J. S., R. H. Galvin, K. M. See, J. Wang, V. Shi. 1995. Functional interactions between YY1 and adenovirus E1A. Nucleic Acids Res. 23:925-31.
33. Lee, J.-S., K. M., Galvin, and Y. Shi. 1993. Evidence for physical interaction between the zinc-finger transcription factors YY1 and Sp1. Proc. Natl. Acad. Sci. USA 90:6145-6149.
34. Lee, J.-S., K. M. Galvin, R. H. See, R. Eckner, D. Livingston, E. Moran, and Y. Shi. 1995. Relief of YY1 transcriptional repression by adenovirus E1A is mediated by E1A-associated protein p300. Genes Dev. 9:1188-1198.
35. Levine, M., and J. L. Manley. 1989. Transcriptional repression of eukaryotic promoters. Cell. 59:405-408.
36. Li, W., H. Y. Chen, & J. R. Davie. 1996. Properties of chicken erythrocyte histone deacetylase associated with the nuclear matrix. Biochem. J. 314: 631-637.
37. Li, Y., J. C. Kappes, J. A. Conway, R. W. Price, G. M. Shaw, and B. H. Hahn. 1991. Complete nucleotide sequence, genomic organization, and biological properties of human immunodeficiency virus type 1 in vivo: evidence for limited defectiveness and complementation. J. Virol. 59:284-291.
38. Lim, L. C., S. L. Swendenmann, and M. Scheffrey. 1992. Molecular cloning of the α-globin transcription factor CP2. Mol. Cell. Bio. 12:828-835.
39. Magnaghi-Jaulin, L., R. Groisman, I. Naguibneva, P. Robin, S. Lorain, J. P. Le Villain, F. Troalen, D. Trouche, & A. Harel-Bellan. 1997. Retinoblastoma protein represses transcription by recruiting a histone deacetylase. Nature (London) 391: 601-605.
40. Malim, M. H., R. Fenrick, D. W. Ballard, J. Hauber, E. Bohnlein, and B. R. Cullen. 1989. Functional characterization of a complex protein-DNA-binding domain located within the human immunodeficiency virus type 1 long terminal repeat leader region. J. Virol. 63:3213-9.
41. Margolis, D. M., M. Somasundaran, and M. R. Green, 1994. Human transcription factor YY1 represses human immunodeficiency virus type 1 transcription and virion production. J. Virol. 68: 905-910.
42. Nagy, L., H.-Y. Kao, D. Chakravarti, R. J. Lin, C. A. Hassig, D. E. Ayer, S. L. Schreiber, & R. M. Evans. 1997. Nuclear receptor repression mediated by a complex containing SMRT, mSin3A, and histone deacetylase. Cell. 89: 373-380.
43. Nan, X., H. H. Ng, C. A. Johnson, C. D. Laherty, B. M. Turner, R. N. Eisenman, and A. Bird. 1998. Transcriptional repression by the methyl-CpG-binding protein MeCP2 involves a histone deacetylase complex. Nature. 393: 386-389.
44. Parada, C. A., J. B. Yoon, and R. G. Roeder. 1995. A novel LBP-1-mediated restriction of HIV-1 transcription at the level of elongation in vitro. J. Biol. Chem. 270; 2274-83.
45. Park, K. and M. L. Atchison. 1991. Isolation of a candidate repressor/activator, NF-E1 (YY-1, delta), that binds to the immunoglobulin kappa 3′ enhancer and the immunoglobulin heavy-chain mu E1 site. Proc. Natl. Acad. Sci. USA. 88: 9804-9808.
46. Pazin, M. J., P. L. Sheridan, K. Cannon, Z. Cao, J. G. Keck, J. T. Kadonaga, & K. A. Jones. 1996. NF-kappa B-mediated chromatin reconfiguration and transcriptional activation of the HIV-1 enhancer in vitro. Genes Dev. 10: 37-49.
47. Pazin, M. J., and J. T. Kadonaga. 1997. What's up and down with histone deacetylation and transcription? Cell. 89:325-328.
48. Ratnasabapathy, R., M. Sheldon, L. Johal, and N. Hernandez. 1990. The HIV-1 long terminal repeat contains an unusual element that induces the synthesis of short RNAs from various mRNA & snRNA promoters. Genes Dev. 4. 2061-2074.
49. Romerio, F., M. N. Gabriel, and D. M. Margolis. 1997. Repression of human immunodeficiency virus type 1 through the novel cooperation of human factors YY1 and LSF. J. Virol. 71: 9375-9382.
50. Seto, E., B. Lewis, and T. Shenk. 1993. Interaction between transcription factors Sp1 and YY1. Nature (London). 365: 462-464.
51. Sheridan, P. L., T. P. Mayall, E. Verdin, K. A. Jones. 1997. Histone acetyltransferases regulate HIV-1 enhancer activity in vitro. Genes & Devel. 11: 3327-3340.
52. Shi, Y., J. S. Lee, and K. M. Galvin. 1997. Everything you have ever wanted to know about Yin Yang 1. 1997. Biochim Biophys Acta. 1332:F49-66.
53. Shi, Y., E. Seto, L.-S. Chang, and T. Shenk. 1991. Transcriptional repression by YY1, a human GLI-Jrippel-related protein, and relief of repression by adenovirus E1A protein. Cell. 67: 377-388.
54. Shirra, M. K., and U. Hansen. 1998. LSF and NTF-1 Share a Conserved DNA Recognition Motif yet Require Different Oligomerization States to Form a Stable Protein-DNA Complex. J. Biol. Chem. 273: 19260-19268.
55. Shirra, M. K., Q. Zhu, H.-C. Huang, D. Pallas, and U. Hansen. 1994. One exon of the human LSF gene includes conserved regions involved in novel DNA-binding and dimerization motifs. Mol. Cell. Biol. 14: 5076-5087.
56. Shrivastava, A., and K. Calame. 1994. An analysis of genes regulated by the multi-functional transcriptional regulator Yin Yang-1. Nucleic Acids Res. 22:5151-5.
57. Shrivastava, A., S. Saleque, G. V. Kalpana, S. Artandi, S. P. Goft and K. Calame. 1993. Inhibition of transcriptional regulator Yin-Yang-1 by association with c-Myc. Science. 262: 1889-1892.
58. Struhl, K. 1996. Chromatin structure and RNA polymerase II connection: implications for transcription. Cell. 84: 179-182.
58b. Sun, J. M., H. Y. Chen, M. Moniwa, S. Samuel, and J. R. Davie. 1999. Purification and characterization of chicken erythrocyte histone deacetylase 1. Biochemistry 38: 5939-47.
59. Taunton, J., C. A. Hassig, and S. L. Schreiber. 1996. A mammalian histone deacetylase related to the yeast transcriptional regulator Rpd3p. Science. 272: 408-411.
60. Usheva, A. and T. Shenk. 1994. TATA-binding protein-independent initiation: YY1, TFIIB, and RNA polymerase II direct basal transcription on supercoiled template DNA. Cell. 76:1115-2182.
61. Van Lint, C., S. Ermiliani, M. Ott, and E. Verdin. 1996. Transcriptional activation and chromatin remodeling of the HIV-1 promoter in response to histone acetylation. EMBO Journal. 15 (5): 1112-1120.
62. Verdin, E. 1991. DNase I-hypersensitive sites are associated with both long terminal repeats and with the intragenic enhancer of integrated human immunodeficiency virus type 1. J. Virol. 65 (12): 6790-6799.
63. Verdin, E., P. Paras, Jr., C. Van Lint. 1993. Chromatin disruption in the promoter of human immunodeficiency virus type 1 during transcriptional activation. EMBO Journal. 12 (8): 3249-3259.
64. Volker, J. L., L. E. Rameh, Q. Zhu, J. DeCaprio, and U. Hansen. 1997. Mitogenic stimulation of resting T cells causes rapid phosphorylation of the transcription factor LSF and increased DNA-binding activity. Genes Dev. 11: 1435-1446.
65. Webster, L. B. and R. P. Ricciardi. 1991. Trans-dominant mutants of E1A provide genetic evidence that the zinc finger of the trans-activating domain binds a transcription factor. Mol Cell Biol. 11: 4287-4296.
66. Williams R. S., S. A. Johnston, M. Riedy, M. J. DeVit, S. G. McElligot, and J. C. Sanford. 1991. Introduction of foreign genes into tissues of living mice by DNA-coated microprojectiles. Proc Natl Acad Sci USA 88: 2726-2730.
67. Wolffe, A. P. 1992. New insights into chromatin function in transcriptional control. FASEB J. 6: 3354-3361.
68. Wong, J. K., M. Hezareh, H. F. Gunthard, D. V. Havlit, C. I. Ignacio, C. A. Spina, D. D. Richman. (1997). Recovery of replication-competent HIV despite prolonged suppression of plasma viremia. Science 278:1291-1295.
69. Wu, F. K., J. A. Garcia, D. Harrich, and R. B, Gaynor. 1988. Purification of the human immunodeficiency virus type 1 enhancer and TAR binding proteins EBP-1 and UBP-1. EMBO J. 7:2117-30.
70. Yang W M, C. Inouye, and E. Seto, 1995. Cyclophilin A and FKBPP12 interact with YY1 and alter its transcriptional activity. J. Biol. Chem. 270: 15187-15193.
71. Yang W M, C. Inouye, Y. Zeng, D. Bearss, and E. Seto. 1996. Transcriptional repression by YY1 is mediated by interaction with a mammalian homolog of the yeast global regulator RPD3. Proc. Natl. Acad. Sci. USA. 93: 12845-12850.
72. Yang W M, Y L Yao, J M Sun, J R Davie, and E. Seto. 1997. Isolation and characterization of cDNAs corresponding to an additional member of the human histone deacetylase gene family. J. Biol. Chem. 272: 28001-28007.
73. Coon, J.-B., G. Li, and R. G. Roeder. 1994. Characterization of a family of related cellular transcription factors which can modulate human immunodeficiency virus type 1 transcription in vitro. Mol. Cell. Biol. 14: 1776-1785.
74. Zhang, Y., R. Iratni, H. Bromage-Erdjument, P. Tempst, & D. Reinberg. 1997. Histone deacetylases and SAP18, a novel polypeptide, are components of a human Sin3 complex. Cell. 89: 357-364.
75. Zhong, F., S. L. Swendeman, W. Popik, P. M. Pitha, and M. Sheffery. 1994. Evidence that levels of the dimeric cellular transcription factor CP2 play little role in the activation of the HIV-1 long terminal repeat in vivo or following superinfection with herpes simplex virus type 1. J. Biol. Chem. 269: 21269-21276.
76. Zhou Q., R. W. Gendrich, and D. A. Engel. 1995. Transcriptional repression of the c-fos gene by YY1 is mediated by a direct interaction with ATF/CREB. J. Virol. 69:4323-4330.

What is claimed is:

1-23. (canceled)

24. A method of treating latent HIV infection in a human subject in need of such treatment, comprising administering to the subject:
a) an amount of an inhibitor of HDAC1 activity, said amount being effective to inhibit repression of HIV transcription, and
b) a therapeutically effective amount of one or more anti-retroviral drugs.


SEQUENCE LISTING (7 sequences)

SEQ ID NO: 1 - DNA, 200 bp, Human immunodeficiency virus
caagggactttccgctggggactttccagggaggcgtggcctgggcgggactggggagtggcgagccctcagatgctgcatataagcagctgctttttgcctgtactgggtctctctggttagaccagatttgagcctgggagctctctggctaactagggaacccactgcttaagcctcaataaagcttgccttgagtg

SEQ ID NO: 2 - DNA, 2353 bp, Homo sapiens
  CDS (241)..(1482)
cgccgagacgagcagcggccgagcgagcgcgggcgcgggcgcaccgaggcgagggaggcggggaagccccgccgccgccgccccgcccgccccttcccccgccgcccgccccctctccccccgcccgctcgccgccttcctccctctgccttccttccccacggccggccgcctcctcgcccgcccgcccgcagccgaggagccgaggccgccgcggccgtggcggcggagccctcagccatggcctcgggcgacaccctctacatcgccacggacggctcggagatgccggccgagatcgtggagctgcacgagatcgaggtggagaccatcccggtggagaccatcgagaccacagtggtgggcgaggaggaggaggaggacgacgacgacgaggacggcggcggtggcgaccacggcggcgggggcggccacgggcacgccggccaccaccaccaccaccatcaccaccaccaccacccgcccatgatcgctctgcagccgctggtcaccgacgacccgacccaggtgcaccaccaccaggaggtgatcctggtgcagacgcgcgaggaggtggtgggcggcgacgactcggacgggctgcgcgccgaggacggcttcgaggatcagattctcatcccggtgcccgcgccggccggcggcgacgacgactacattgaacaaacgctggtcaccgtggcggcggccggcaagagcggcggcggcggctcgtcgtcgtcgggaggcggccgcgtcaagaagggcggcggcaagaagagcggcaagaagagttacctcagcggcggggccggcgcggcgggcgggcgcggcgccgacccgggcaacaagaagtgggagcagaagcaggtgcagatcaagaccctggagggcgagttctcggtcaccatgtggtcctcagatgaaaaaaaagatattgaccatgagacagtggttgaagaacagatcattggagagaactcacctcctgattattcagaatatatgacaggaaagaaacttcctcctggaggaatacctggcattgacctctcagatcccaaacaactggcagaatttgctagaatgaagccaagaaaaattaaagaagatgatgctccaagaacaatagcttgccctcataaaggctgcacaaagatgttcagggataactcggccatgagaaaacatctgcacacccacggtcccagagtccacgtctgtgcagaatgtggcaaagcttttgttgagagttcaaaactaaaacgacaccaactggttcatactggagagaagccctttcagtgcacgttcgaaggctgtgggaaacgcttttcactggacttcaatttgcgcacacatgtgcgaatccataccggagacaggccctatgtgtgccccttcgatggttgtaataagaagtttgctcagtcaactaacctgaaatctcacatcttaacacatgctaaggccaaaaacaaccagtgaaaagaagagagaagacccttctcgaccacgggaagcatcttccagaagtgtgattgggaataaatatgcctctcctttgtatattatttctaggaagaattttaaaaatgaatcctacacacctaagggacatgttttgataaagtagtaaaaattaaaaaaaaaaaactttactaagatgacattgctaagatgctctatcttgctctgtaatctcgtttcaaaaacacagtgtttttgtaaagtgtggtcccaacaggaggacaattcatgaacttcgcatcaaaagacaattctttatacaacagtgctaaaaatgggacttcttttcacattcttataaatatgaagctcacctgttgcttacaatttttttaattttgtattttccaagtgtgcatattgtacacttttttggggatatgcttagtaatgctacgtgtgatttttctggaggttgataactttgcttgcagtagattttctttaaaagaatgggcagttacatgcatacttcaaaagtattttcctgtaaaaaaaaaaaaagttatataggttttgtttgctatcttaattttggttgtattctttgatgttaacacattttgtataattgtatcgtatagctgtattgaatcatgtagtatcaaatattagatgtgatttaatagtgttaatcaatttaaacccattttagtcactttttttttccaaaaaaatactgccagatgctgatgttcagtgtaatttctttgcctgttcagttacagaaagtggtgctcagttgtagaatgtattgtaccttttaacacctgatgtgtacatcccatgtaacagaaagggcaacaataaaatagcaatcctaaag

SEQ ID NO: 3 - protein, 414 aa, Homo sapiens [sequence omitted]

SEQ ID NO: 4 - DNA, 2418 bp, Homo sapiens
  CDS (692)..(2197)
gaggacgccatgattggttggcgctggggcggcggacggtggaagggcctggcgagtctaggttttacgcctgtgctggactttctccttccatgtttccaggccgtggggggctacagagggcgagaagtcggctcagcggaaacctggatttggttctaagccgtggggttgagaaggggtgaccggaagtgatcgtgggactgaccggaagcgaggcctggaggggaaagagagagcgagacctgggagggagggggcctccagcagaaaggggcgggggaaaaggtgcaaaagcagcgtgggagcgccgggctggcttcctgcggctgctgctggtctgactgggaagcagcaagccaccactacgaactctcaagaggagtgggagtgcgggagtccagagctgcctctgggaagtctgcagtagttgagcaaaggggtcctcacgttcctgagagctgggcaggggggattttggaacctggggcagccaagaacgagcagccaagggtacgggagattagttgtgcacagagcagtgctggtcgggcttgggggtggctggtgggcactgcgtgggaaaccttggtttgtagttttcttggtttgcgttactcctgttgggtagaattaccctccgcgcctttgtacaagacacggtgtctcctggggcaaggaaggagccaggatggcctgggctctgaagctgcctctggccgacgaagtgattgaatccgggttggtgcaggactttgatgctagcctgtccgggatcggccaggaactgggtgctggtgcctatagcatgagtgatgtccttgcattgcccatttttaagcaagaagagtcgagtttgcctcctgataatgagaataaaatcctgccttttcaatatgtgctttgtgctgctacctctccagcagtgaaactccatgatgaaaccctaacgtatctcaatcaaggacagtcttatgaaattcgaatgctagacaataggaaacttggagaacttccagaaattaatggcaaattggtgaagagtatattccgtgtggtgttccatgacagaaggcttcagtacactgagcatcagcagctagagggctggaggtggaaccgacctggagacagaattcttgacatagatatcccgatgtctgtgggtataatcgatcctagggctaatccaactcaactaaatacagtggagttcctgtgggaccctgcaaagaggacatctgtgtttattcaggtgcactgtattagcacagagttcactatgaggaaacatggcggagaaaagggggtgccattccgagtacaaatagataccttcaaggagaatgaaaacggggaatatactgagcacttacactcggccagctgccagatcaaagttttcaagcccaaaggtgcagacagaaagcaaaaaacggatagggaaaaaatggagaaacgaacacctcatgaaaaggagaaatatcagccttcctatgagacaaccatactcacagagtgttctccatggcccgagatcacgtatgtcaataactccccatcacctggcttcaacagttcccatagcagtttttctcttggggaaggaaatggttcaccaaaccaccagccagagccaccccctccagtcacagataacctcttaccaacaaccacacctcaggaagctcagcagtggttgcatcgaaatcgtttttctacattcacaaggcttttcacaaacttctcaggggcagatttattgaaattaactagagatgatgtgatccaaatctgtggccctgcagatggaatcagactttttaatgcattaaaaggccggatggtgcgtccaaggttaaccatttatgtttgtcaggaatcactgcagttgagggagcagcaacaacagcagcagcaacagcagcagaagcatgaggatggagactcaaatggtactttcttcgtttaccatgctatctatctagaagaactaacagctgttgaattgacagaaaaaattgctcagcttttcagcatttccccttgccagatcagccagatttacaagcaggggccaacaggaattcatgtgctcatcagtgatgagatgatacagaactttcaggaagaagcatgttttattctggacacaatgaaacaggaaaccaatgatagctatcatatcatactgaagtaggagtgcggcgtttcgtgcccagtggctgctccttccttcacctctgaaaacggccctcttgaagggggatatgaatggagatttgaaggtctgcaagaacctgactcgtctgactgtgtgtggaggagtccaggccatggaggcagaatcctggccctctgtgttggcccaagctcttgtggtacacacagagggccaggattctgcctccatggcct

SEQ ID NO: 5 - protein, 502 aa, Homo sapiens [sequence omitted]

SEQ ID NO: 6 - protein, 482 aa, Homo sapiens [sequence omitted]

SEQ ID NO: 7 - DNA, 2091 bp, Homo sapiens
gagcggagccgcgggcgggagggcggacggaccgactgacggtagggacgggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgcaggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttcttccccaacccctcagattttatattttctatttctctgtgtatttatataaaaatttattaaatataaatatccccagggacagaaaccaaggccccgagctcagggcagctgtgctgggtgagctcttccaggagccaccttgccacccattcttcccgttcttaactttgaaccataaagggtgccaggtctgggtgaaagggatacttttatgcaaccataagacaaactcctgaaatgccaagtgcctgcttagtagctttggaaaggtgcccttattgaacattctagaaggggtggctgggtcttcaaggatctcctgtttttttcaggctcctaaagtaacatcagccatttttagattggttctgttttcgtaccttcccactggcctcaagtgagccaagaaacactgcctgccctctgtctgtcttctcctaattctgcaggtggaggttgctagtctagtttcctttttgagatactattttcatttttgtgagcctctttgtaataaaatggtacatttct